(19) United States
(12) Patent Application Publication
(10) Pub. No.: US 2009/0203034 A1
(43) Pub. Date: Aug. 13, 2009

(54) Reagents for the detection of tyrosine phosphorylation in brain ischemia signaling pathways

(76) Inventors: Ailan Guo, Burlington, MA (US); Roberto Polakiewicz, Lexington, MA (US); Kimberly Lee, Seattle, WA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(21) Appl. No.: 12/227,318

(22) PCT Filed: May 11, 2007
(86) PCT No.: PCT/US2007/011418

§ 371 Date: Nov. 12, 2008

Related U.S. Application Data
(60) Provisional application No. 60/799,963, filed on May 12, 2006.

(51) Int. Cl. G01N 33/53; C12Q 1/48; G01N 33/536; C07K 16/18; C07K 14/47; C07K 7/06; C07K 7/08; C12N 5/16

(52) U.S. Cl. 435/7.1; 435/15; 436/536; 530/389.8; 530/324; 530/328; 530/327; 530/326; 435/326; 530/387.9

(57) ABSTRACT

The invention discloses 99 novel phosphorylation sites identified in signal transduction proteins and pathways underlying human Brain Ischemia, and provides phosphorylation-site specific antibodies and heavy-isotope labeled peptides (AQUA peptides) for the selective detection and quantification of these phosphorylated sites/proteins, as well as methods of using the reagents for such purpose. Among the phosphorylation sites identified are sites occurring in the following protein types: protein kinases, adaptor/scaffold proteins, adhesion proteins, G proteins/GTPase/Guanine nucleotide exchange factors, Calcium binding proteins, cytoskeletal proteins, Channel proteins, Chaperone proteins, Helicases, Motor proteins, Translation proteins, RNA binding proteins, Ubiquitin conjugating system proteins, vesicle proteins and Receptor proteins.

### RELATED APPLICATIONS

This application claims the benefit of, and priority to, U.S. Ser. No. 60/799,963, filed May 12, 2006, presently pending, the disclosure of which is incorporated herein, in its entirety, by reference.

### FIELD OF THE INVENTION

The invention relates generally to antibodies and peptide reagents for the detection of protein tyrosine phosphorylation, and to protein tyrosine phosphorylation associated with brain ischemia.

### BACKGROUND OF THE INVENTION

The activation of proteins by post-translational modification is an important cellular mechanism for regulating most aspects of biological organization and control, including growth, development, homeostasis, and cellular communication. Protein phosphorylation, for example, plays a critical role in the etiology of many pathological conditions and diseases, including cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Manning et al., Science. 298:1912-34 (2002). Most kinases phosphorylate many different substrate proteins, at distinct tyrosine, serine, or threonine residues. Indeed, it is estimated that one-third of all proteins encoded by the human genome are phosphorylated, and many are phosphorylated at multiple sites by different kinases. See Graves et al., Pharmacol. Ther. 82: 111-21 (1999).

Tyrosine phosphorylation plays critical roles in all areas of neurological development and function including stem cell survival and differentiation, axon guidance and synaptogenesis, and synaptic transmission of mature neurons. See Lee and van Vactor, Current Biology, 13, R152-R161 (2003). A number of protein tyrosine kinases and phosphatases, the enzymes that regulate protein tyrosine phosphorylation, have been shown to play essential roles in normal brain growth, development and function. Eph receptors, the largest family of receptor tyrosine kinases, are expressed at high levels in the brain. They not only play significant roles in controlling brain development during embryogenesis but also are critical in regulating excitatory synapses in the adult brain. See Murai and Pasquale, Neuron 33:159-62 (2002). The Trk family of receptor tyrosine kinases, receptors for nerve growth factor and related molecules, is essential for neuronal proliferation, differentiation, growth and survival. See Huang and Reichardt, Annu Rev Biochem. 72:609-42, (2003). The insulin receptor, a receptor tyrosine kinase, plays an important role in memory and associative learning. See Zhao and Alkon, Mol Cell Endocrinol. 177:125-34 (2001). Src, fyn, and Abl are all cytoplasmic tyrosine kinases that are known to modulate brain functions including depolarization, memory and neurogenesis. See Yu et al., Science 275: 674-678 (1997); Kojima et al., Proc. Nat'l. Acad. Sci. USA 94: 4761-4765 (1997); Koleske et al., Neuron 21: 1259-1272 (1998).

Like the tyrosine kinases, many protein tyrosine phosphatases (PTPs) are expressed in the adult brain and are essential to normal brain development and function. See Paul and Lombroso, Cell Mol Life Sci. 60:2465-82 (2003). PTPs are known to play roles in NMDA receptor regulation and attenuating potentiation of NMDA currents. See Ferrani-Kile and Leslie, J Pharmacol Exp Ther. 314:86-93 (2005); Grishin et al., Neuropharmacology 49:328-37 (2005). LAR-RPTP, a receptor protein tyrosine phosphatase, plays a role in the expression of surface AMPA receptors, in targeting of the cadherin-beta-catenin complex, and the development and maintenance of excitatory synapses. See Dunah et al., Nat. Neurosci. 8:458-67 (2005). The PTP Shp2 plays a role in cerebral cortex development as well as controlling energy balance and metabolism in the adult forebrain. See Yamamoto et al. Proc Natl Acad Sci USA. 102:15983-8 (2005) and Zhang et al. Proc Natl Acad Sci USA. 101:16064-9 (2004).

While many specific protein tyrosine kinases and phosphatases are known to be essential to neurological development and function, relatively few of the specific targets and substrates of these enzymes have been identified. Some of the known substrates of protein tyrosine kinases in the brain include the kinases themselves, the Rho GTPase regulator protein p190RhoGAP, GABAA receptors, b-catenin, Enabled, PLC-gamma, IRS-1 and -2, alpha-synuclein, etc. See Pawson, Eur J Cancer 38: Suppl 5:S3-10 (2002); Brouns et al., Nat. Cell Biol. 3: 361-367 (2001); Moss et al., Nature 377: 344-348 (1995); Yamada et al., J Biol Chem 272: 30334-30339 (1997); and Ahn et al., J Biol Chem 277: 12334-12342 (2002). There is strong experimental evidence suggesting that there are thousands of tyrosine phosphorylation sites on proteins in the brain. Understanding the relevance of these phosphorylation sites to normal brain functioning, and the signaling pathways in which they participate, will be crucial for understanding both normal brain function and for intervening in neurological pathologies such as brain ischemia.

Protein tyrosine kinases, phosphatases, and tyrosine-phosphorylated proteins have all been shown to participate in post-ischemia/reperfusion damage and may have implications for the treatment of stroke. Tyrosine phosphorylation of synaptic proteins increases ten- to one hundred-fold after brain ischemia. See Hu et al., J. Neurochem. 62:1357-67 (1994). N-Shc, an adaptor protein that binds a specific tyrosine-phosphorylated site on the receptor tyrosine kinase Ret, is neural-protective following ischemia/reperfusion stress in mice. See Troglio et al., Proc Natl Acad Sci USA. 101:15476-81 (2004). The RPTK VEGFR has been shown to cause edema formation and tissue damage following ischemia/reperfusion injury in the mouse brain. See van Bruggen et al., J Clin Invest. 104:1613-20 (1999). Reactive oxygen species (ROS) released following ischemia/reperfusion injury, causing significant tissue damage. See Rayner et al., J Neurochem. 97(1):211-21 (2006). Tyrosine protein phosphatases are inactivated by ROS, and may contribute to the build up of tyrosine phosphorylated neuronal proteins following ischemia/reperfusion. See Levinthal and Defranco, J Biol. Chem. 280:5875-83 (2005), and Suzaki et al., J Biol. Chem. 277(11):9614-21 (2002).

Inhibition of specific protein kinases following ischemia/reperfusion is a promising strategy for the treatment of stroke. A number of kinase inhibitors administered intravenously following ischemia/reperfusion in animal models have significantly reduced ischemic brain injury. These include U0126, a specific MAPK kinase inhibitor; SP600125, a specific inhibitor of c-Jun N-terminal kinase (JNK); and K252a, an inhibitor of MLK3/MKK7/JNK3. See Namura et al., Proc Natl Acad Sci USA. 98:11569-74 (2001); Guan et al., Brain Res. 1035:51-9 (2005); and Pan et al., Neuroscience. 131:147-59 (2005).

Despite the identification of some protein kinases, phosphatases, and substrates involved in brain ischemic damage, the vast majority of signaling protein changes involved in this disease remains unknown. This paucity of information about the specific proteins and phosphorylation sites impedes our understanding of the role of tyrosine phosphorylation signaling in ischemia/reperfusion injury.

Accordingly, there is a need to discover the tyrosine phosphorylation sites and proteins involved in ischemic damage in order to understand the tyrosine phosphorylation pathways, kinases and phosphatases that contribute to ischemic brain damage. A thorough understanding of the phosphorylation pathways activated during ischemia/reperfusion will help identify specific kinases and phosphatases that are candidates for therapeutic intervention. In addition, research reagents, such as phospho-specific antibodies and AQUA peptides, will be produced that are specific for particular phosphorylation sites. These reagents, which will detect and quantify specific phosphorylation sites involved in the various pathways and stages of ischemic damage, will provide molecular tools for basic research as well as for evaluating the efficacy of experimental therapeutics in animal models.

### SUMMARY OF THE INVENTION

The invention discloses 99 novel phosphorylation sites identified in signal transduction proteins and pathways underlying Brain Ischemia and provides new reagents, including phosphorylation-site specific antibodies and AQUA peptides, for the selective detection and quantification of these phosphorylated sites/proteins. Also provided are methods of using the reagents of the invention for the detection and quantification of the disclosed phosphorylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the brain ischemia signaling protein tyrosine phosphorylation sites disclosed herein:

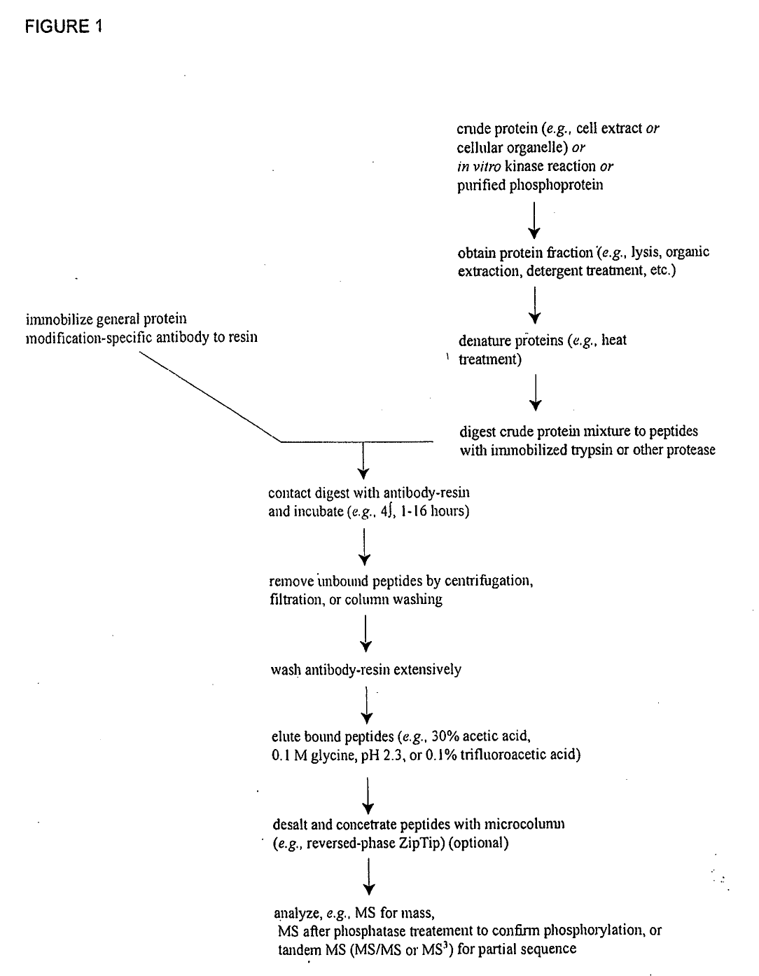

Column A=the name of the parent protein; Column B=the SwissProt accession number for the protein (human sequence); Column C=the protein type/classification; Column D=the tyrosine residue (in the parent protein amino acid sequence) at which phosphorylation occurs within the phosphorylation site; Column E=the phosphorylation site sequence encompassing the phosphorylatable residue (residue at which phosphorylation occurs (and corresponding to the respective entry in Column D) appears in lowercase; Column F=the cell type(s), tissue(s) and/or patient(s) in which the phosphorylation site was discovered; and Column G=The SEQ ID NO which identifies the each phosphorylation site sequence.

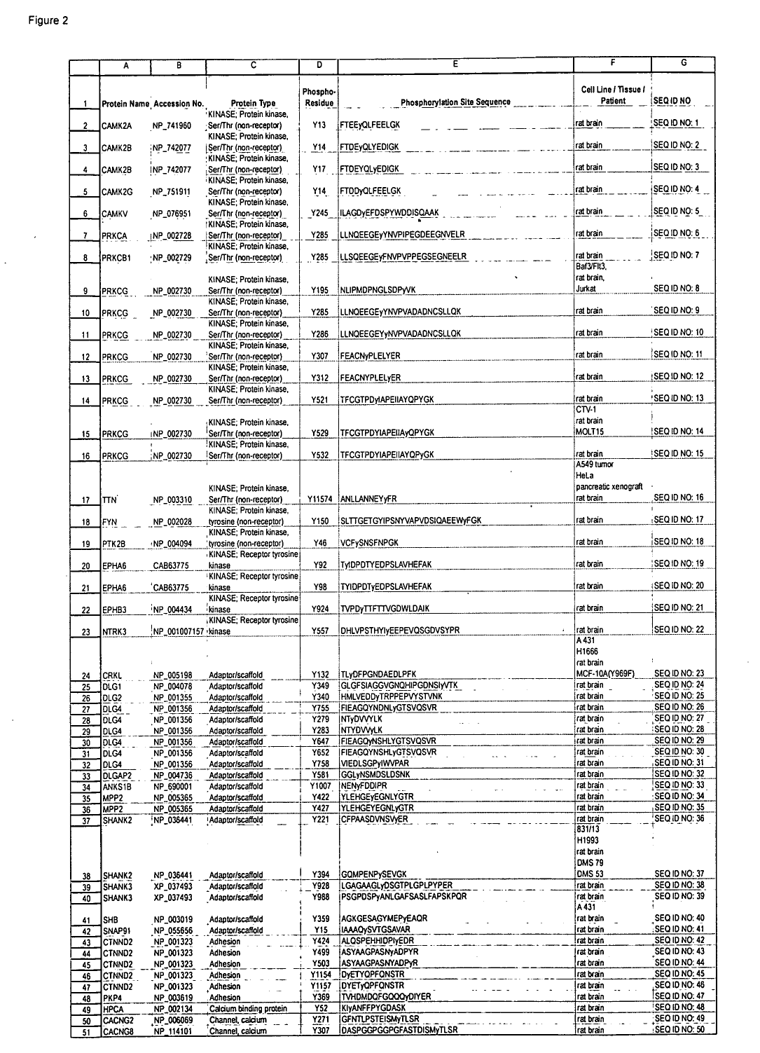

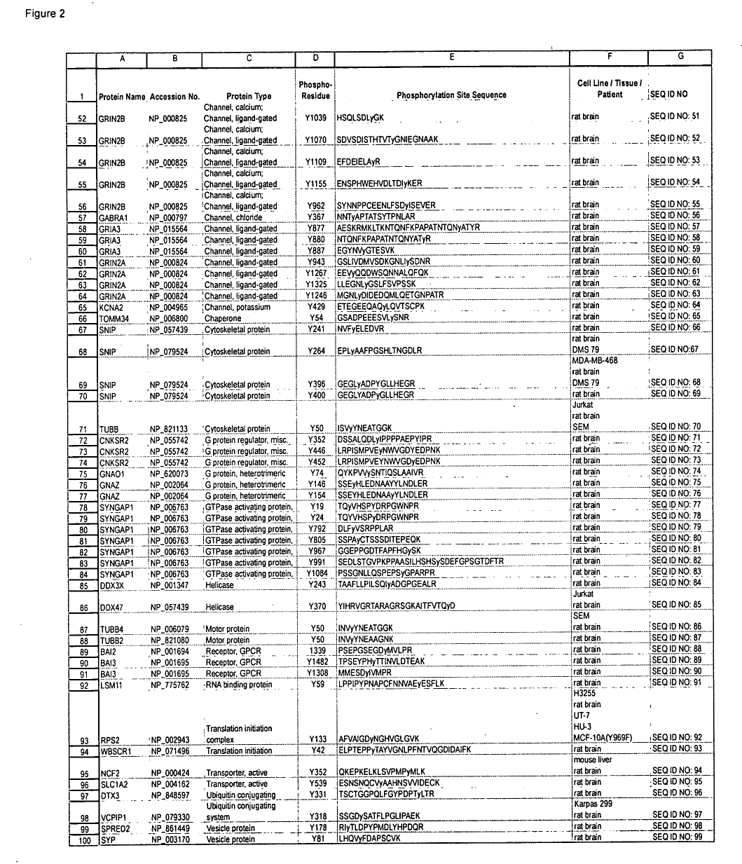

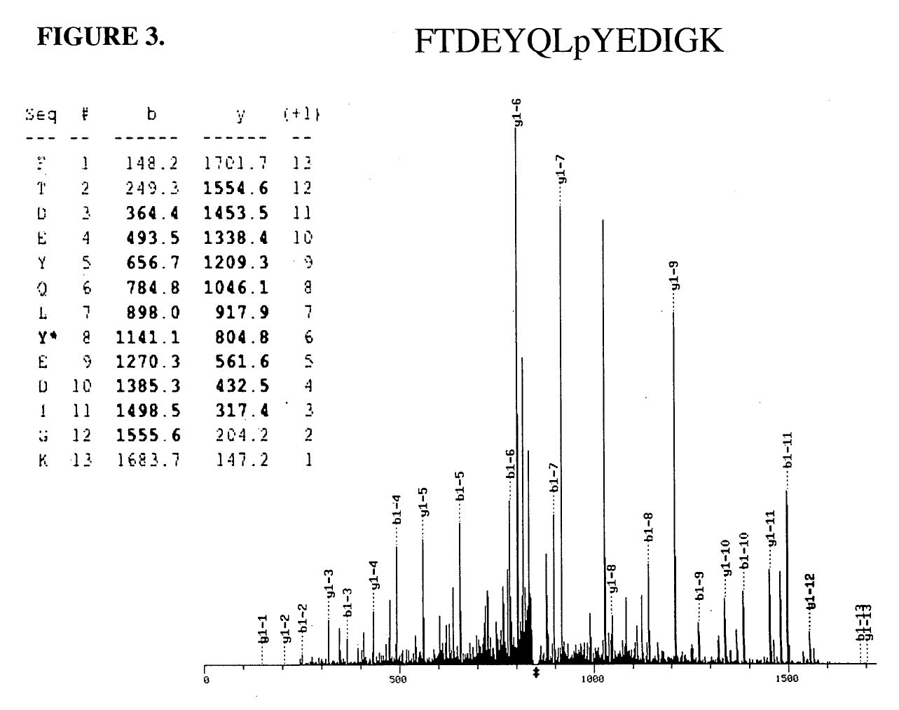

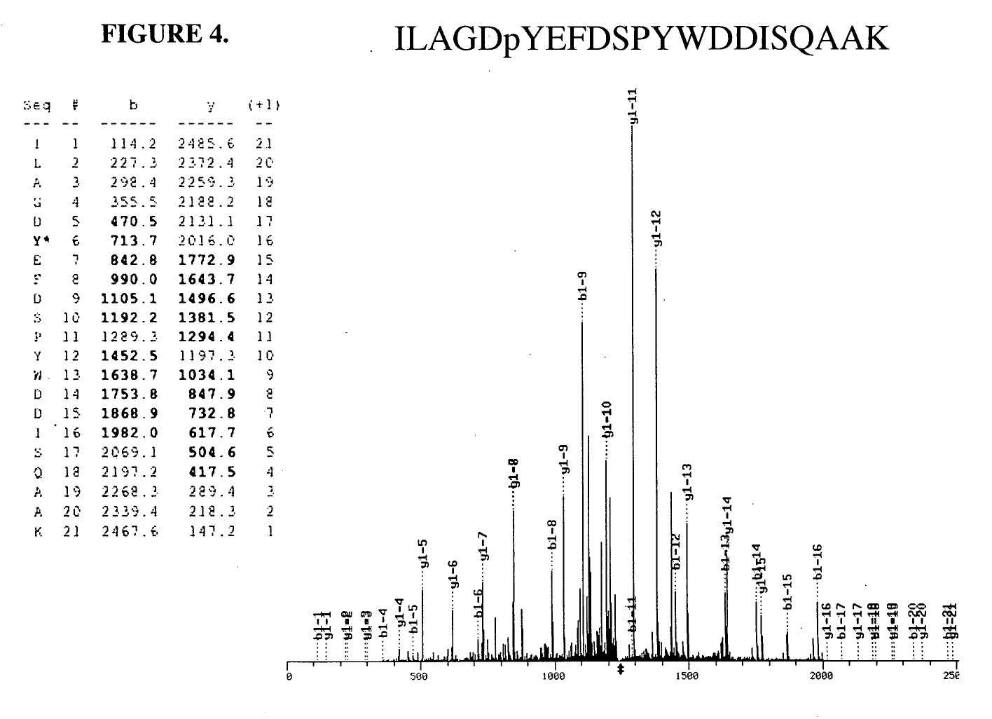

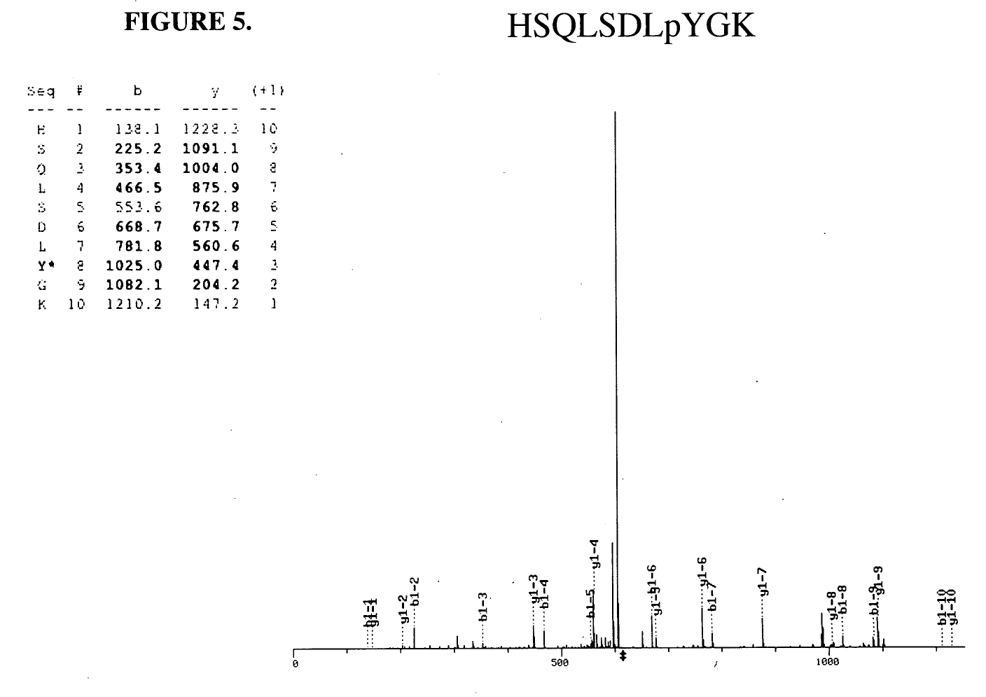

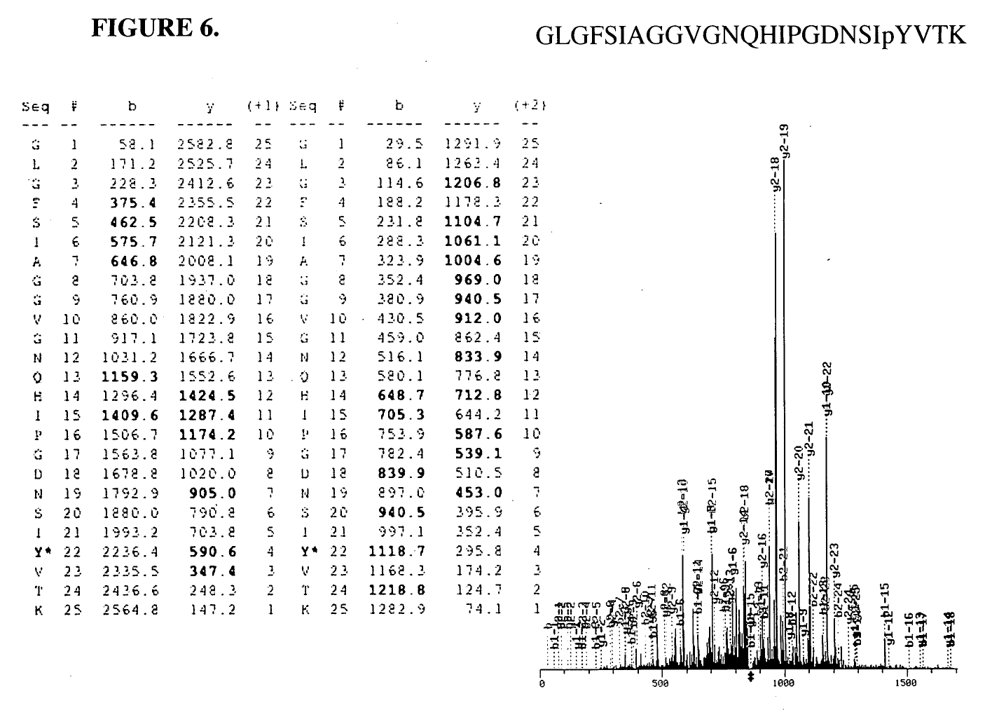

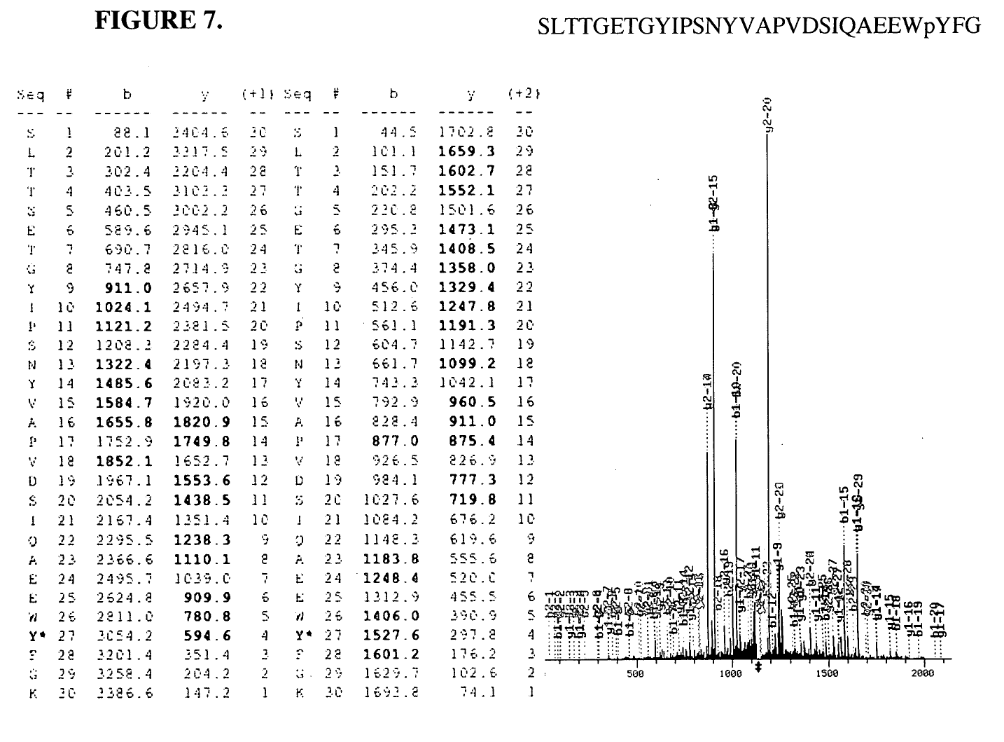

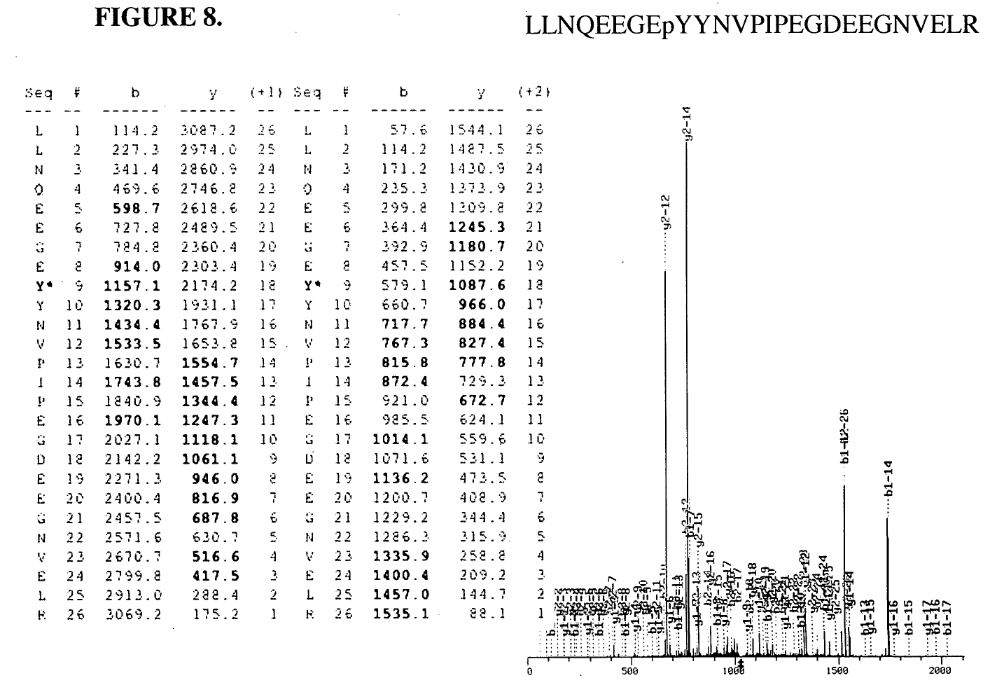

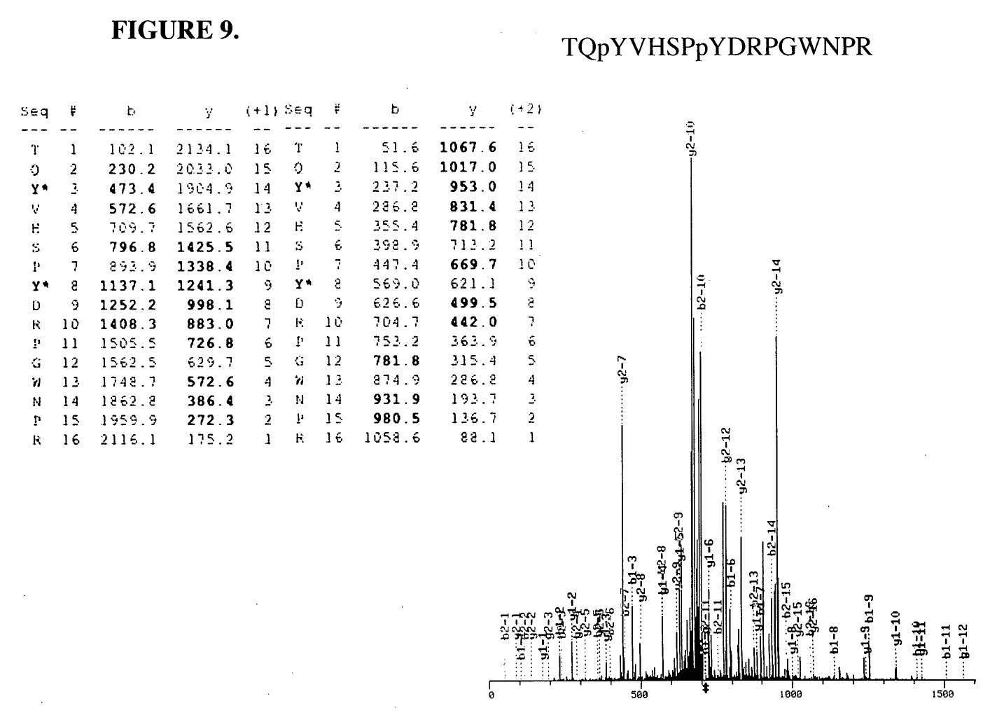

FIG. 3—is an exemplary mass spectrograph depicting the detection of the tyrosine 17 phosphorylation site in CAMK2B (see Row 4 in FIG. 2/Table 1), as further described in Example 1 (red and blue indicate ions detected in MS/MS spectrum); y* indicates the phosphorylated tyrosine (shown as lowercase “y” in FIG. 2).

FIG. 4—is an exemplary mass spectrograph depicting the detection of the tyrosine 245 phosphorylation site in CAMKV (see Row 6 in FIG. 2/Table 1), as further described in Example 1 (red and blue indicate ions detected in MS/MS spectrum); y* indicates the phosphorylated tyrosine (shown as lowercase “y” in FIG. 2).

FIG. 5—is an exemplary mass spectrograph depicting the detection of the tyrosine 1039 phosphorylation site in GRIN2B (see Row 52 in FIG. 2/Table 1), as further described in Example 1 (red and blue indicate ions detected in MS/MS spectrum); y* indicates the phosphorylated serine (shown as lowercase “y” in FIG. 2).

FIG. 6—is an exemplary mass spectrograph depicting the detection of the tyrosine 349 phosphorylation site in rat-brain DLG1 (see Row 25 in FIG. 2/Table 1 for orthologous human site), as further described in Example 1 (red and blue indicate ions detected in MS/MS spectrum); y* indicates the phosphorylated tyrosine (shown as lowercase “y” in FIG. 2).

FIG. 7—is an exemplary mass spectrograph depicting the detection of the tyrosine 150 phosphorylation site in rat-brain FYN (see Row 18 in FIG. 2/Table 1 for orthologous human site), as further described in Example 1 (red and blue indicate ions detected in MS/MS spectrum); y* indicates the phosphorylated tyrosine (shown as lowercase “y” in FIG. 2).

FIG. 8—is an exemplary mass spectrograph depicting the detection of the tyrosine 285 phosphorylation site in rat-brain PRKCA (see Row 7 in FIG. 2/Table 1 for orthologous human site), as further described in Example 1 (red and blue indicate ions detected in MS/MS spectrum); y* indicates the phosphorylated tyrosine (shown as lowercase “y” in FIG. 2).

FIG. 9—is an exemplary mass spectrograph depicting the detection of the tyrosine 19 and 24 phosphorylation sites in rat-brain SYNGAP1 (see Rows 78 and 79 in FIG. 2/Table 1 for orthologous human site), as further described in Example 1 (red and blue indicate ions detected in MS/MS spectrum); y* indicates the phosphorylated tyrosine (shown as lowercase “y” in FIG. 2).

### DETAILED DESCRIPTION OF THE INVENTION

In accordance with the present invention, 99 novel protein phosphorylation sites in signaling proteins and pathways underlying human Brain Ischemia have now been discovered. These newly described phosphorylation sites were identified by employing the techniques described in “Immunoaffinity Isolation of Modified Peptides From Complex Mixtures,” U.S. Patent Publication No. 20030044848, Rush et al., using cellular extracts from a variety of cell lines, e.g. rat brain, DMS 53 etc., as further described below. The novel phosphorylation sites (tyrosine), and their corresponding parent proteins were originally found in rat brain and the orthologous site in human was identified using Homologene at NCBI. These orthologus human sites disclosed herein are listed in Table 1. These phosphorylation sites correspond to numerous different parent proteins (the full sequences (human) of which are all publicly available in SwissProt database and their Accession numbers listed in Column B of Table 1/FIG. 2), each of which fall into discrete protein type groups, for example adhesion proteins, adaptor/scaffold proteins, cytoskeletal proteins, protein kinases, and DNA binding proteins, etc. (see Column C of Table 1), the phosphorylation of which is relevant to signal transduction activity underlying Brain Ischemia, as disclosed herein.

The discovery of the 99 novel protein phosphorylation sites described herein enables the production, by standard methods, of new reagents, such as phosphorylation site-specific antibodies and AQUA peptides (heavy-isotope labeled peptides), capable of specifically detecting and/or quantifying these phosphorylated sites/proteins. Such reagents are highly useful, inter alia, for studying signal transduction events underlying the progression of Brain Ischemia. Accordingly, the invention provides novel reagents—phospho-specific antibodies and AQUA peptides—for the specific detection and/or quantification of a Brain Ischemia-related signaling protein/polypeptide only when phosphorylated (or only when not phosphorylated) at a particular phosphorylation site disclosed herein. The invention also provides methods of detecting and/or quantifying one or more phosphorylated Brain Ischemia-related signaling proteins using the phosphorylation-site specific antibodies and AQUA peptides of the invention.

In part, the invention provides an isolated phosphorylation site-specific antibody that specifically binds a given Brain Ischemia-related signaling protein only when phosphorylated (or not phosphorylated, respectively) at a particular tyrosine enumerated in Column D of Table 1/FIG. 2 comprised within the phosphorylatable peptide site sequence enumerated in corresponding Column E. In further part, the invention provides a heavy-isotope labeled peptide (AQUA peptide) for the detection and quantification of a given Brain Ischemia-related signaling protein, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the CRKL adaptor/scaffold protein only when phosphorylated (or only when not phosphorylated) at tyrosine 132 (see Row 24 (and Columns D and E) of Table 1/FIG. 2). By way of further example, among the group of reagents provided by the invention is an AQUA peptide for the quantification of phosphorylated TUBB cytoskeletal protein, the AQUA peptide comprising the phosphorylatable peptide sequence listed in Column E, Row 71, of Table 1/FIG. 2 (which encompasses the phosphorylatable tyrosine at position 50).

In one embodiment, the invention provides an isolated phosphorylation site-specific antibody that specifically binds a human Brain Ischemia-related signaling protein selected from Column A of Table 1 (Rows 2-100) only when phosphorylated at the tyrosine residue listed in corresponding Column D of Table 1, comprised within the phosphorylatable peptide sequence listed in corresponding Column E of Table 1 (SEQ ID NOs: 1-99), wherein said antibody does not bind said signaling protein when not phosphorylated at said tyrosine. In another embodiment, the invention provides an isolated phosphorylation site-specific antibody that specifically binds a Brain Ischemia-related signaling protein selected from Column A of Table 1 only when not phosphorylated at the tyrosine residue listed in corresponding Column D of Table 1, comprised within the peptide sequence listed in corresponding Column E of Table 1 (SEQ ID NOs: 1-99), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one preferred embodiment, the immortalized cell line is a rabbit or mouse hybridoma.

In another embodiment, the invention provides a heavy-isotope labeled peptide (AQUA peptide) for the quantification of a Brain Ischemia-related signaling protein selected from Column A of Table 1, said labeled peptide comprising the phosphorylatable peptide sequence listed in corresponding Column E of Table 1 (SEQ ID NOs: 1-99), which sequence comprises the phosphorylatable tyrosine listed in corresponding Column D of Table 1. In certain preferred embodiments, the phosphorylatable tyrosine within the labeled peptide is phosphorylated, while in other preferred embodiments, the phosphorylatable residue within the labeled peptide is not phosphorylated.

Reagents (antibodies and AQUA peptides) provided by the invention may conveniently be grouped by the type of Brain Ischemia-related signaling protein in which a given phosphorylation site (for which reagents are provided) occurs. The protein types for each respective protein (in which a phosphorylation site has been discovered) are provided in Column C of Table 1/FIG. 2, and include: protein kinases, adaptor/scaffold proteins, adhesion proteins, G proteins/GTPase/Guanine nucleotide exchange factors, Calcium binding proteins, cytoskeletal proteins, Channel proteins, Chaperone proteins, Helicases, Motor proteins, Translation proteins, RNA binding proteins, Ubiquitin conjugating system proteins, vesicle proteins and Receptor proteins. Each of these distinct protein groups is considered a preferred subset of Brain Ischemia-related signal transduction protein phosphorylation sites disclosed herein, and reagents for their detection/quantification may be considered a preferred subset of reagents provided by the invention.

Particularly preferred subsets of the phosphorylation sites (and their corresponding proteins) disclosed herein are those occurring on the following protein types/groups listed in Column C of Table 1/FIG. 2 Protein kinases, Adaptor/scaffold proteins, Channel Proteins, G proteins/GTPase/Guanine nucleotide exchange factors, Cytoskeletal proteins, and Adhesion proteins. Accordingly, among preferred subsets of reagents provided by the invention are isolated antibodies and AQUA peptides useful for the detection and/or quantification of the foregoing preferred protein/phosphorylation site subsets.

In one subset of preferred embodiments, there is provided:

(i) An isolated phosphorylation site-specific antibody that specifically binds a Protein kinase selected from Column A, Rows 2-23, of Table 1 only when phosphorylated at the tyrosine listed in corresponding Column D, Rows 2-23, of Table 1, comprised within the phosphorylatable peptide sequence listed in corresponding Column E, Rows 2-23, of Table 1 (SEQ ID NOs: 1-22), wherein said antibody does not bind said protein when not phosphorylated at said tyrosine.
(ii) An equivalent antibody to (i) above that only binds the Protein kinase when not phosphorylated at the disclosed site (and does not bind the protein when it is phosphorylated at the site).
(iii) A heavy-isotope labeled peptide (AQUA peptide) for the quantification of a Protein kinase selected from Column A, Rows 2-23, said labeled peptide comprising the phosphorylatable peptide sequence listed in corresponding Column E, Rows 2-23, of Table 1 (SEQ ID NOs: 1-22), which sequence comprises the phosphorylatable tyrosine listed in corresponding Column D, Rows 2-23, of Table 1.

Among this preferred subset of reagents, antibodies and AQUA peptides for the detection/quantification of the following Protein kinase phosphorylation sites are particularly preferred: CAMK2B (Y17), CAMKV (Y245), PRKCA (Y245), PRKCB1 (Y285) and FYN (Y150) (see SEQ ID NOs: 3, 5, 6, 7 and 17).

In a second subset of preferred embodiments there is provided:

(i) An isolated phosphorylation site-specific antibody that specifically binds an Adaptor/scaffold protein selected from Column A, Rows 24-42, of Table 1 only when phosphorylated at the tyrosine listed in corresponding Column D, Rows 24-42, of Table 1, comprised within the phosphorylatable peptide sequence listed in corresponding Column E, Rows 24-42, of Table 1 (SEQ ID NOs: 23-41), wherein said antibody does not bind said protein when not phosphorylated at said tyrosine.
(ii) An equivalent antibody to (i) above that only binds the Adaptor/scaffold protein when not phosphorylated at the disclosed site (and does not bind the protein when it is phosphorylated at the site).
(iii) A heavy-isotope labeled peptide (AQUA peptide) for the quantification of a Brain Ischemia-related signaling protein that is a Adaptor/scaffold protein selected from Column A, Rows 24-42, said labeled peptide comprising the phosphorylatable peptide sequence listed in corresponding Column E, Rows 24-42, of Table 1 (SEQ ID NOs: 2341), which sequence comprises the phosphorylatable tyrosine listed in corresponding Column D, Rows 24-42, of Table 1.

Among this preferred subset of reagents, antibodies and AQUA peptides for the detection/quantification of the following Adaptor/scaffold protein phosphorylation sites are particularly preferred: CRKL (Y132), DLG1 (Y349), SHANK2 (Y221) and SHB (Y359) (see SEQ ID NOs: 24, 25, 37 and 41).

In another subset of preferred embodiments there is provided:

(i) An isolated phosphorylation site-specific antibody that specifically binds a Channel protein selected from Column A, Rows 50-65, of Table 1 only when phosphorylated at the tyrosine listed in corresponding Column D, Rows 50-65, of Table 1, comprised within the phosphorylatable peptide sequence listed in corresponding Column E, Rows 50-65, of Table 1 (SEQ ID NOs: 49-64), wherein said antibody does not bind said protein when not phosphorylated at said tyrosine.
(ii) An equivalent antibody to (i) above that only binds the Channel protein when not phosphorylated at the disclosed site (and does not bind the protein when it is phosphorylated at the site).
(iii) A heavy-isotope labeled peptide (AQUA peptide) for the quantification of a Brain Ischemia-related signaling protein that is an Channel protein selected from Column A, Rows 50-65, said labeled peptide comprising the phosphorylatable peptide sequence listed in corresponding Column E, Rows 50-65, of Table 1 (SEQ ID NOs: 49-64), which sequence comprises the phosphorylatable tyrosine listed in corresponding Column D, Rows 50-65, of Table 1.

Among this preferred subset of reagents, antibodies and AQUA peptides for the detection/quantification of the following Channel protein phosphorylation sites are particularly preferred: GRIN2B (Y1039), GRIN2B (Y1070), GABRA1 (Y367), GRIA3 (Y877), GRIN2A (Y943), GRIN2A (Y1246) and KCNA2 (Y429) (see SEQ ID NO's: 51, 52, 56, 57, 60, 63, and 64).

In still another subset of preferred embodiments there is provided:

(i) An isolated phosphorylation site-specific antibody that specifically binds a Cytoskeletal protein selected from Column A, Rows 67-71, of Table 1 only when phosphorylated at the tyrosine listed in corresponding Column D, Rows 67-71, of Table 1, comprised within the phosphorylatable peptide sequence listed in corresponding Column E, Rows 67-71, of Table 1 (SEQ ID NOs: 66-70), wherein said antibody does not bind said protein when not phosphorylated at said tyrosine.
(ii) An equivalent antibody to (i) above that only binds the Cytoskeletal protein when not phosphorylated at the disclosed site (and does not bind the protein when it is phosphorylated at the site).
(iii) A heavy-isotope labeled peptide (AQUA peptide) for the quantification of a Brain Ischemia-related signaling protein that is a Cytoskeletal protein selected from Column A, Rows 67-71, said labeled peptide comprising the phosphorylatable peptide sequence listed in corresponding Column E, Rows 67-71, of Table 1 (SEQ ID NOs: 66-70), which sequence comprises the phosphorylatable tyrosine listed in corresponding Column D, Rows 67-71, of Table 1.

In still another subset of preferred embodiments there is provided:

(i) An isolated phosphorylation site-specific antibody that specifically binds an Adhesion protein selected from Column A, Rows 43-48, of Table 1 only when phosphorylated at the tyrosine listed in corresponding Column D, Rows 43-48, of Table 1, comprised within the phosphorylatable peptide sequence listed in corresponding Column E, Rows 43-48, of Table 1 (SEQ ID NOs: 42-47), wherein said antibody does not bind said protein when not phosphorylated at said tyrosine.
(ii) An equivalent antibody to (i) above that only binds the Adhesion protein when not phosphorylated at the disclosed site (and does not bind the protein when it is phosphorylated at the site).
(iii) A heavy-isotope labeled peptide (AQUA peptide) for the quantification of a Brain Ischemia-related signaling protein that is an Adhesion protein selected from Column A, Rows 43-48, said labeled peptide comprising the phosphorylatable peptide sequence listed in corresponding Column E, Rows 43-48, of Table 1 (SEQ ID NOs: 42-47), which sequence comprises the phosphorylatable tyrosine listed in corresponding Column D, Rows 43-48, of Table 1.

In still another subset of preferred embodiments there is provided:

(i) An isolated phosphorylation site-specific antibody that specifically binds a G protein/GTPase/Guanine nucleotide exchange factor selected from Column A, Rows 72-84, of Table 1 only when phosphorylated at the tyrosine listed in corresponding Column D, Rows 72-84, of Table 1, comprised within the phosphorylatable peptide sequence listed in corresponding Column E, Rows 72-84 of Table 1 (SEQ ID NOs: 71-83), wherein said antibody does not bind said protein when not phosphorylated at said tyrosine.
(ii) An equivalent antibody to (i) above that only binds G protein/GTPase/Guanine nucleotide exchange factor when not phosphorylated at the disclosed site (and does not bind the protein when it is phosphorylated at the site).
(iii) A heavy-isotope labeled peptide (AQUA peptide) for the quantification of a Brain Ischemia-related signaling protein that is a G protein/GTPase/Guanine nucleotide exchange factor selected from Column A, Rows 72-84, said labeled peptide comprising the phosphorylatable peptide sequence listed in corresponding Column E, Rows 72-84, of Table 1 (SEQ ID NOs: 71-83), which sequence comprises the phosphorylatable tyrosine listed in corresponding Column D, Rows 72-84, of Table 1.

Among this preferred subset of reagents, antibodies and AQUA peptides for the detection/quantification of the following G protein/GTPase/Guanine nucleotide exchange factor phosphorylation sites are particularly preferred: GNAZ (Y146), GNAZ (Y154), SYNGAP1 (Y19) and SYNGAP (Y24) (see SEQ ID NOs: 75, 76, 77 and 78).

The invention also provides, in part, an immortalized cell line producing an antibody of the invention, for example, a cell line producing an antibody within any of the foregoing preferred subsets of antibodies. In one preferred embodiment, the immortalized cell line is a rabbit hybridoma or a mouse hybridoma.

In certain other preferred embodiments, a heavy-isotope labeled peptide (AQUA peptide) of the invention (for example, an AQUA peptide within any of the foregoing preferred subsets of AQUA peptides) comprises a disclosed site sequence wherein the phosphorylatable tyrosine is phosphorylated. In certain other preferred embodiments, a heavy-isotope labeled peptide of the invention comprises a disclosed site sequence wherein the phosphorylatable tyrosine is not phosphorylated.

The foregoing subsets of preferred reagents of the invention should not be construed as limiting the scope of the invention, which, as noted above, includes reagents for the detection and/or quantification of disclosed phosphorylation sites on any of the other protein type/group subsets (each a preferred subset) listed in Column C of Table 1/FIG. 2.

Also provided by the invention are methods for detecting or quantifying a Brain Ischemia-related signaling protein that is tyrosine phosphorylated, said method comprising the step of utilizing one or more of the above-described reagents of the invention to detect or quantify one or more Brain Ischemia-related signaling protein(s) selected from Column A of Table 1 only when phosphorylated at the tyrosine listed in corresponding Column D of Table 1. In certain preferred embodiments of the methods of the invention, the reagents comprise a subset of preferred reagents as described above.

The identification of the disclosed novel Brain Ischemia-related signaling protein phosphorylation sites, and the standard production and use of the reagents provided by the invention are described in further detail below and in the Examples that follow.

All cited references are hereby incorporated herein, in their entirety, by reference. The Examples are provided to further illustrate the invention, and do not in any way limit its scope, except as provided in the claims appended hereto.

TABLE 1
Newly Discovered Brain Ischemia-related Phosphorylation Sites.

| | A | B | | D | | |
|---|---|---|---|---|---|---|
| | Protein | Accession | C | Phospho- | E | G |
| 1 | Name | No. | Protein Type | Residue | Phosphorylation Site Sequence | SEQ ID NO |

| | | | | | | |
|---|---|---|---|---|---|---|
| 2 | CAMK2A | NP_741960 | KINASE; Protein | Y13 | FTEEyQLFEELGK | SEQ ID NO: 1 |
| | | | kinase, Ser/Thr (non- | | | |
| | | | receptor) | | | |
| 3 | CAMK2B | NP_742077 | KINASE; Protein | Y14 | FTDEyQLYEDIGK | SEQ ID NO: 2 |
| | | | kinase, Ser/Thr (non- | | | |
| | | | receptor) | | | |
| 4 | CAMK2B | NP_742077 | KINASE; Protein | Y17 | FTDEYQLyEDIGK | SEQ ID NO: 3 |
| | | | kinase, Ser/Thr (non- | | | |
| | | | receptor) | | | |
| 5 | CAMK2G | NP_751911 | KINASE; Protein | Y14 | FTDDyQLFEELGK | SEQ ID NO: 4 |
| | | | kinase, Ser/Thr (non- | | | |
| | | | receptor) | | | |
| 6 | CAMKV | NP_076951 | KINASE; Protein | Y245 | ILAGDyEFDSPYWDDISQAAK | SEQ ID NO: 5 |
| | | | kinase, Ser/Thr (non- | | | |
| | | | receptor) | | | |
| 7 | PRKCA | NP_002728 | KINASE; Protein | Y285 | LLNQEEGEyYNVPIPEGDEEGNVELR | SEQ ID NO: 6 |
| | | | kinase, Ser/Thr (non- | | | |
| | | | receptor) | | | |
| 8 | PRKCB1 | NP_002729 | KINASE; Protein | Y285 | LLSQEEGEyFNVPVPPEGSEGNEELR | SEQ ID NO: 7 |
| | | | kinase, Ser/Thr (non- | | | |
| | | | receptor) | | | |
| 9 | PRKCG | NP_002730 | KINASE; Protein | Y195 | NLIPMDPNGLSDPyVK | SEQ ID NO: 8 |
| | | | kinase, SerlThr (non- | | | |
| | | | receptor) | | | |
| 10 | PRKCG | NP_002730 | KINASE; Protein | Y285 | LLNQEEGEyYNVPVADADNCSLLQK | SEQ ID NO: 9 |
| | | | kinase, Ser/Thr (non- | | | |
| | | | receptor) | | | |
| 11 | PRKCG | NP_002730 | KINASE; Protein | Y286 | LLNQEEGEYyNVPVADADNCSLLQK | SEQ ID NO: 10 |
| | | | kinase, Ser/Thr (non. | | | |
| | | | receptor) | | | |
| 12 | PRKCG | NP_002730 | KINASE; Protein | Y307 | FEACNyPLELYER | SEQ ID NO: 11 |
| | | | kinase, Ser/Thr (non- | | | |
| | | | receptor) | | | |
| 13 | PRKCG | NP_002730 | KINASE; Protein | Y312 | FEACNYPLELyER | SEQ ID NO: 12 |
| | | | kinase, Ser/Thr (non | | | |
| | | | receptor) | | | |
| 14 | PRKCG | NP_002730 | KINASE; Protein | Y521 | TFCGTPDyIAPEIIAYQPYGK | SEQ ID NO: 13 |
| | | | kinase, Ser/Thr (non | | | |
| | | | receptor) | | | |
| 15 | PRKCG | NP_002730 | KINASE; Protein | Y529 | TFCGTPDYIAPEIIAyQPYGK | SEQ ID NO: 14 |
| | | | kinase, Ser/Thr (non- | | | |
| | | | receptor) | | | |
| 16 | PRKCG | NP_002730 | KINASE; Protein | Y532 | TFCGTPDYIAPEIIAYQPyGK | SEQ ID NO: 15 |
| | | | kinase, Ser/Thr (non- | | | |
| | | | receptor) | | | |
| 17 | TTN | NP_003310 | KINASE; Protein | Y11574 | ANLLANNEYyFR | SEQ ID NO: 16 |
| | | | kinase, Ser/Thr (non- | | | |
| | | | receptor) | | | |
| 18 | FYN | NP_002028 | KINASE; Protein | Y150 | SLTTGETGYIPSNYVAPVDSIQAEEWyFG | SEQ ID NO: 17 |
| | | | kinase, tyrosine (non- | | K | |
| | | | receptor) | | | |
| 19 | PTK2B | NP_004094 | KINASE; Protein | Y46 | VCFySNSFNPGK | SEQ ID NO: 18 |
| | | | kinase, tyrosine (non- | | | |
| | | | receptor) | | | |
| 20 | EPHA6 | CAB63775 | KINASE; Receptor | Y92 | TyIDPDTYEDPSLAVHEFAK | SEQ ID NO: 19 |
| | | | tyrosine kinase | | | |
| 21 | EPHA6 | CAB63775 | KINASE; Receptor | Y98 | TYIDPDTyEDPSLAVHEFAK | SEQ ID NO: 20 |
| | | | tyrosine kinase | | | |
| 22 | EPHB3 | NP_004434 | KINASE; Receptor | Y924 | TVPDyTTFTTVGDWLDAIK | SEQ ID NO: 21 |
| | | | tyrosine kinase | | | |
| 23 | NTRK3 | NP_001007157 | KINASE; Receptor | Y557 | DHLVPSTHYIyEEPEVQSGDVSYPR | SEQ ID NO: 22 |
| | | | tyrosine kinase | | | |
| 24 | CRKL | NP_005198 | Adaptor/scaffold | Y132 | TLyDFPGNDAEDLPFK | SEQ ID NO: 23 |
| 25 | DLG1 | NP_004078 | Adaptor/scaffold | Y349 | GLGFSIAGGVGNQHIPGDNSIyVTK | SEQ ID NO: 24 |
| 26 | DLG2 | NP_001355 | Adaptor/scaffold | Y340 | HMLVEDDyTRPPEPVYSTVNK | SEQ ID NO: 25 |
| 27 | DLG2 | NP_001356 | Adaptor/scaffold | Y755 | FIEAGQYNDNLyGTSVQSVR | SEQ ID NO: 26 |
| 28 | os | NP_001356 | Adaptor/scaffold | Y279 | NTyDVVYLK | SEQ ID NO: 27 |
| 29 | DLG4 | NP_001356 | Adaptor/scaffold | Y283 | NTYDWyLK | SEQ ID NO: 28 |
| 30 | DLG4 | NP_001356 | Adaptor/scaffold | Y647 | FIEAGQyNSHLYGTSVQSVR | SEQ ID NO: 29 |
| 31 | DLG4 | NP_001356 | Adaptor/scaffold | Y652 | FIEAGQYNSHLyGTSVQSVR | SEQ ID NO: 30 |
| 32 | DLG4 | NP_001356 | Adaptor/scaffold | Y758 | VIEDLSGPyIWVPAR | SEQ ID NO: 31 |
| 33 | DLGAP2 | NP_004736 | Adaptor/scaffold | Y581 | GGLyNSMDSLDSNK | SEQ ID NO: 32 |
| 34 | ANKS1B | NP_690001 | Adaptor/scaffold | Y1007 | NENyFDDIPR | SEQ ID NO: 33 |
| 35 | MPP2 | NP_005365 | Adaptor/scaffold | Y422 | YLEHGEyEGNLYGTR | SEQ ID NO: 34 |
| 36 | MPP2 | NP_005365 | Adaptor/scaffold | Y427 | YLEHGEYEGNLyGTR | SEQ ID NO: 35 |
| 37 | SHANK2 | NP_036441 | Adaptor/scaffold | Y221 | CFPAASDVNSVyER | SEQ ID NO: 36 |
| 38 | SHANK2 | NP_036441 | Adaptor/scaffold | Y394 | GQMPENPySEVGK | SEQ ID NO: 37 |
| 39 | SHANK3 | XP_037493 | Adaptor/scaffold | Y928 | LGAGAAGLyDSGTPLGPLPYPER | SEQ ID NO: 38 |
| 40 | SHANK3 | XP_037493 | Adaptor/scaffold | Y988 | PSGPDSPyANLGAFSASLFAPSKPQR | SEQ ID NO: 39 |
| 41 | SHB | NP_003019 | Adaptor/scaffold | Y359 | AGKGESAGYMEPyEAQR | SEQ ID NO: 40 |
| 42 | SNAP91 | NP_055656 | Adaptor/scaffold | Y15 | IAAAQySVTGSAVAR | SEQ ID NO: 41 |
| 43 | CTNND2 | NP_001323 | Adhesion | Y424 | ALQSPEHHIDPIyEDR | SEQ ID NO: 42 |
| 44 | CTNND2 | NP_001323 | Adhesion | Y499 | ASYAAGPASNyADPYR | SEQ ID NO: 43 |
| 45 | CTNND2 | NP_001323 | Adhesion | Y503 | ASYAAGPASNYADPyR | SEQ ID NO: 44 |
| 46 | CTNND2 | NP_001323 | Adhesion | Y1154 | DyETYQPFQNSTR | SEQ ID NO: 45 |
| 47 | CTNND2 | NP_001323 | Adhesion | Y1157 | DYETyQPFQNSTR | SEQ ID NO: 46 |
| 48 | PKP4 | NP_003619 | Adhesion | Y369 | TVHDMDQFGQQQyDIYER | SEQ ID NO: 47 |
| 49 | HPCA | NP_002134 | Calcium binding protein | Y52 | KIyANFFPYGDASK | SEQ ID NO: 48 |
| 50 | CACNG2 | NP_006069 | Channel, calcium | Y271 | GFNTLPSTEISMyTLSR | SEQ ID NO: 49 |
| 51 | CACNG8 | NP_14101 | Channel, calcium | Y307 | DASPGGPGGPGFASTDISMyTLSR | SEQ ID NO: 50 |
| 52 | GRIN2B | NP_000825 | Channel, calcium; | | | |
| | | | Channel, ligand-gated | Y1039 | HSQLSDLyGK | SEQ ID NO: 51 |
| 53 | GRIN2B | NP_000825 | Channel, calcium; | | | |
| | | | Channel, ligand-gated | Y1070 | SDVSDISTHTVTyGNIEGNMK | SEQ ID NO: 52 |
| 54 | GRIN2B | NP_000825 | Channel, calcium; | | | |
| | | | Channel, ligand-gated | Y1109 | EFDEIELAyR | SEQ ID NO: 53 |
| 55 | GRIN2B | NP_000825 | Channel, calcium; | | | |
| | | | Channel, ligand-gated | Y1155 | ENSPHWEHVDLTDIyKER | SEQ ID NO: 54 |
| 56 | GRIN2B | NP_000825 | Channel, calcium; | | | |
| | | | Channel, ligand-gated | Y962 | SYNNPPCEENLFSDyISEVER | SEQ ID NO: 55 |
| 57 | GABRA1 | NP_000797 | Channel, chloride | Y367 | NNTyAPTATSYTPNLAR | SEQ ID NO: 56 |
| 58 | GRIA3 | NP_015564 | Channel, ligand-gated | Y877 | AESKRMKLTKNTQNFKPAPATNTQNyAT | SEQ ID NO: 57 |
| | | | | | YR | |
| 59 | GRIA3 | NP_015564 | Channel, ligand-gated | Y880 | NTQNFKPAPATNTQNYATyR | SEQ ID NO: 58 |
| 60 | GRIA3 | NP_015564 | Channel, Iigand-gated | Y887 | EGYNVyGTESVK | SEQ ID NO: 59 |
| 61 | GRIN2A | NP_000824 | Channel, ligand-gated | Y943 | GSLIVDMVSDKGNLIySDNR | SEQ ID NO: 60 |
| 62 | GRIN2A | NP_000824 | Channel, ligand-gated | Y1267 | EEVyQQDWSQNNALQFQK | SEQ ID NO: 61 |
| 63 | GRIN2A | NP_000824 | Channel, ligand-gated | Y1325 | LLEGNLyGSLFSVPSSK | SEQ ID NO: 62 |
| 64 | GRIN2A | NP_000824 | Channel, ligand-gated | Y1246 | MGNLyDIDEDQMLQETGNPATR | SEQ ID NO: 63 |
| 65 | KCNA2 | NP_004965 | Channel, potassium | Y429 | ETEGEEQAQyLQVTSCPK | SEQ ID NO: 64 |
| 66 | TOMM34 | NP_006800 | Chaperone | Y54 | GSADPEEESVLySNRSEQ ID NO: 65 | |
| 67 | SNIP | NP_057439 | Cytoskeletal protein | Y241 | NVFyELEDVR | SEQ ID NO: 66 |
| 68 | SNIP | NP_079524 | Cytoskeletal protein | Y264 | EPLyAAFPGSHLTNGDLR | SEQ ID NO: 67 |
| 69 | SNIP | NP_079524 | Cytoskeletal protein | Y396 | GEGLyADPYGLLHEGR | SEQ ID NO: 68 |
| 70 | SNIP | NP_079524 | Cytoskeletal protein | Y400 | GEGLYADPyGLLHEGR | SEQ ID NO: 69 |
| 71 | TUBB | NP_821133 | Cytoskeletal protein | Y50 | ISVyYNEATGGK | SEQ ID NO: 70 |
| 72 | CNKSR2 | NP_055742 | G protein regulator, | Y352 | DSSALQDLyIPPPPAEPYIPR | SEQ ID NO: 71 |
| | | | misc. | | | |
| 73 | CNKSR2 | NP_055742 | G protein regulator, | Y˜6 | LRPISMPVEyNWVGDYEDPNK | SEQ ID NO: 72 |
| | | | misc. | | | |
| 74 | CNKSR2 | NP_055742 | G protein regulator, | Y452 | LRPISMPVEYNWVGDyEDPNK | SEQ ID NO: 73 |
| | | | misc. | | | |
| 75 | GNAO1 | NP_620073 | G protein, | Y74 | QYKPWySNTIQSLAAIVR | SEQ ID NO: 74 |
| | | | heterotnmeric | | | |
| 76 | GNAZ | NP_002064 | G protein, | Y146 | SSEyHLEDNMYYLNDLER | SEQ ID NO: 75 |
| | | | heterotrimeric | | | |
| 77 | GNAZ | NP_002064 | G protein, | Y154 | SSEYHLEDNMyYLNDLER | SEQ ID NO: 76 |
| | | | heterotrimeric | | | |
| 78 | SYNGAP1 | NP_006763 | GTPase activating | Y19 | TQyVHSPYDRPGWNPR | SEQ ID NO: 77 |
| | | | protein, Ras | | | |
| 79 | SYNGAP1 | NP_006763 | GTPase activating | Y24 | TQYVHSPyDRPGWNPR | SEQ ID NO: 78 |
| | | | protein, Ras | | | |
| 80 | SYNGAP1 | NP_006763 | GTPase activating | Y792 | DLFyVSRPPLAR | SEQ ID NO: 79 |
| | | | protein, Ras | | | |
| 81 | SYNGAP1 | NP_006763 | GTPase activating | Y805 | SSPAyCTSSSDITEPEQK | SEQ ID NO: 80 |
| | | | protein, Ras | | | |
| 82 | SYNGAP1 | NP_006763 | GTPase activating | Y967 | GGEPPGDTFAPFHGySK | SEQ ID NO: 81 |
| | | | protein, Ras | | | |
| 83 | SYNGAP1 | NP_006763 | GTPase activating | Y991 | SEDLSTGVPKPPAASILHSHSySDEFGPS | SEQ ID NO: 82 |
| | | | protein, Ras | | GTDFTR | |
| 84 | SYNGAP1 | NP_006763 | GTPase activating | Y1084 | PSSGNLLQSPEPSyGPARPR | SEQ ID NO: 83 |
| | | | protein, Ras | | | |
| 85 | DDX3X | NP_001347 | Helicase | Y243 | TAAFLLPILSQIyADGPGEALR | SEQ ID NO: 84 |
| 86 | DDX47 | NP_057439 | Helicase | Y370 | YIHRVGRTARAGRSGKAITFVTQyD | SEQ ID NO: 85 |
| 87 | TUBB4 | NP_006079 | Motor protein | Y50 | INVyYNEATGGK | SEQ ID NO: 86 |
| 88 | TUBB2 | NP_821080 | Motor protein | Y50 | INVyYNEAAGNK | SEQ ID NO: 87 |
| 89 | BA12 | NP_001694 | Receptor, GPCR | 1339 | PSERGSEGDyMVLPR | SEQ ID NO: 88 |
| 90 | BA13 | NP_001695 | Receptor, GPCR | Y1482 | TPSEYPHyTTINVLDTEAK | SEQ ID NO: 89 |
| 91 | BA13 | NP_001695 | Receptor, GPCR | Y1308 | MMESDyIVMPR | SEQ ID NO: 90 |
| 92 | LSM11 | NP_275762 | RNA binding protein | Y59 | LPPIPYPNAPCFNNVAEyESFLK | SEQ ID NO: 91 |
| 93 | RPS2 | NP_002943 | Translation initiation | Y133 | AFVAIGDyNGHVGLGVK | SEQ ID NO: 92 |
| | | | complex | | | |
| 94 | WBSCR1 | NP_071496 | Translation initiation | Y42 | ELPTEPPyTAYVGNLPFNTVQGDIDAIFK | SEQ ID NO: 93 |
| | | | complex | | | |
| 95 | NCF2 | NP_000424 | Transporter, active | Y352 | QKEPKELKLSVPMPYMLK | SEQ ID NO: 94 |
| 96 | SLC1A2 | NP_004162 | Transporter, active | Y539 | ESNSNQCVyMHNSWIDECK | SEQ ID NO: 95 |
| | | | Ubiquitin conjugating | | | |
| 97 | DTX3 | NP_848597 | system | Y331 | TSCTGGPQLFGYPDPTyLTR | SEQ ID NO: 96 |
| | | | Ubiguitin conjugating | | | |
| 98 | VCPIP1 | NP_079330 | system | Y318 | SSGDySATFLPGLIPAEK | SEQ ID NO: 97 |
| 99 | SPRED2 | NP_861449 | Vesicle protein | Y178 | RIyTLDPYPMDLYHPDQR | SEQ ID NO: 98 |
| 100 | SYP | NP_003170 | Vesicle protein | Y81 | LHQVyFDAPSCVK | SEQ ID NO: 99 |

The short name for each protein in which a phosphorylation site has presently been identified is provided in Column A, and its SwissProt accession number (human) is provided Column B. The protein type/group into which each protein falls is provided in Column C. The identified tyrosine residue at which phosphorylation occurs in a given protein is identified in Column D, and the amino acid sequence of the phosphorylation site encompassing the tyrosine residue is provided in Column E (lower case y=the tyrosine (identified in Column D)) at which phosphorylation occurs. Table 1 above is identical to FIG. 2, except that the latter includes the cell type(s) in which the particular phosphorylation site was identified (Column F). Column G gives the SEQ ID NO for each phosphorylation site.

The identification of these 99 phosphorylation sites is described in more detail in Part A below and in Example 1.

### DEFINITIONS

As used herein, the following terms have the meanings indicated:

“Antibody” or “antibodies” refers to all types of immunoglobulins, including IgG, IgM, IgA, IgD, and IgE, including F_{ab} or antigen-recognition fragments thereof, including chimeric, polyclonal, and monoclonal antibodies. The term “does not bind” with respect to an antibody's binding to one phospho-form of a sequence means does not substantially react with as compared to the antibody's binding to the other phospho-form of the sequence for which the antibody is specific.

“Brain Ischemia-related signaling protein” means any protein (or polypeptide derived therefrom) enumerated in Column A of Table 1/FIG. 2, which is disclosed herein as being phosphorylated in one or more Brain Ischemia cell line(s). Brain Ischemia-related signaling proteins may be tyrosine kinases, such as TTN or BCR, or serine/threonine kinases, or direct substrates of such kinases, or may be indirect substrates downstream of such kinases in signaling pathways. A Brain Ischemia-related signaling protein may also be phosphorylated in other cell lines harboring activated kinase activity.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

### A. Identification of Novel Brain Ischemia-related Protein Phosphorylation Sites.

The 99 novel Brain Ischemia-related signaling protein phosphorylation sites disclosed herein and listed in Table 1/FIG. 2 were discovered by employing the modified peptide isolation and characterization techniques described in “Immunoaffinity Isolation of Modified Peptides From Complex Mixtures,” U.S. patent Publication No. 20030044848, Rush et al. (the teaching of which is hereby incorporated herein by reference, in its entirety) using cellular extracts from the following cell lines and patient samples: rat brain, Baf3/Flt3, Jurkat, CTV-1, MOLT15, A549 tumor, HeLa pancreatic xenograft, A431, H1666, MCF-10A (Y969F), HER4-JMb, 3T3-wt, 3T3-Src, 831/13, KNM-3, CTV-1, Baf3/E255K, Baf3/M351T, Baf3/TpoR, 3T3-EGFRwt, 3T3-EGFR(L858R), TG4, BaF3-Tel/FGFR3, BaF3-TDII, UT-7, HER4-JMa, 3T3-Abl, H1993, DMS 79, DMS 53, MDA-MB-468, SEM, H3255, HU-3, MCF-10A (Y969F), mouse liver and Karpas 299. The isolation and identification of phosphopeptides from these cell lines, using an immobilized general phosphotyrosine-specific antibody, described in detail in Example 1 below. In addition to the 99 previously unknown protein phosphorylation sites (tyrosine) discovered, many known phosphorylation sites were also identified (not described herein). The immunoaffinity/mass spectrometric technique described in the '848 patent Publication (the “IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g. Sequest) may be utilized to substantially match the spectra obtained for the isolated, modified peptide during the characterization of step (d) with the spectra for a known peptide sequence. A quantification step employing, e.g. SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat #9411 (p-Tyr-100)) was used in the immunoaffinity step to isolate the widest possible number of phospho-tyrosine containing peptides from the cell extracts.

Extracts from the following human Brain Ischemia cell lines and patient samples were employed: rat brain, Baf3/Flt3, Jurkat, CTV-1, MOLT15, A549 tumor, HeLa pancreatic xenograft, A431, H1666, MCF-10A (Y969F), HER4-JMb, 3T3-wt, 3T3-Src, 831/13, KNM-3, CTV-1, Baf3/E255K, Baf3/M351T, Baf3/TpoR, 3T3-EGFRwt, 3T3-EGFR(L858R), TG4, BaF3-Tel/FGFR3, BaF3-TDII, UT-7, HER4—JMa, 3T3-Abl, H1993, DMS 79, DMS 53, MDA-MB-468, SEM, H3255, HU-3, MCF-10A (Y969F), mouse liver and Karpas 299.

As described in more detail in the Examples, lysates were prepared and digested with trypsin after treatment with DTT and iodoacetamide to alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in MOPS IP buffer and treated with phosphotyrosine (P-Tyr-100, CST #9411) immobilized on protein G-Sepharose or Protein A-Sepharose. Immunoaffinity-purified peptides were eluted with 0.1% TFA and a portion of this fraction was concentrated with Stage or Zip tips and analyzed by LC-MS/MS, using a ThermoFinnigan LTQ ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed a total of 99 novel tyrosine phosphorylation sites in signaling pathways affected by kinase activation or active in Brain Ischemia cells. The identified phosphorylation sites and their parent proteins are enumerated in Table 1/FIG. 2. The tyrosine at which phosphorylation occurs is provided in Column D, and the peptide sequence encompassing the phosphorylatable tyrosine residue at the site is provided in Column E. All phospho-tyrosine sites were originally found in rat-brain and subsequently the orthologous site in human was identified using Homologene at NCBI; the sequence reported in column E is the phosphorylation site flanked by 7 amino acids on each side. FIG. 2 also shows the particular cell line(s) (see Column F) in which a particular phosphorylation site was discovered.

As a result of the discovery of these phosphorylation sites, phospho-specific antibodies and AQUA peptides for the detection of and quantification of these sites and their parent proteins may now be produced by standard methods, described below. These new reagents will prove highly useful in, e.g., studying the signaling pathways and events underlying the progression of Brain Ischemias and the identification of new biomarkers and targets for diagnosis and treatment of such diseases.

### B. Antibodies and Cell Lines

Isolated phosphorylation site-specific antibodies that specifically bind a Brain Ischemia-related signaling protein disclosed in Column A of Table 1 only when phosphorylated (or only when not phosphorylated) at the corresponding amino acid and phosphorylation site listed in Columns D and E of Table 1/FIG. 2 may now be produced by standard antibody production methods, such as anti-peptide antibody methods, using the phosphorylation site sequence information provided in Column E of Table 1. For example, a previously unknown CRKL adaptor/scaffold phosphorylation site (tyrosine 132) (see Rows 24 of Table 1/FIG. 2) is presently disclosed. Thus, an antiboy that specifically binds this novel CRKL adaptor/scaffold site can now be produced, e.g. by immunizing an animal with a peptide antigen comprising all or part of the amino acid sequence encompassing the respective phosphorylated residue (e.g. a peptide antigen comprising the sequence set forth in Row 24, Column E, of Table 1, SEQ ID NO: 23) (which encompasses the phosphorylated tyrosine at position 132 in CRKL, to produce an antibody that only binds CRKL adaptor/scaffold when phosphorylated at that site.

Polyclonal antibodies of the invention may be produced according to standard techniques by immunizing a suitable animal (e.g., rabbit, goat, etc.) with a peptide antigen corresponding to the Brain Ischemia-related phosphorylation site of interest (i.e. a phosphorylation site enumerated in Column E of Table 1, which comprises the corresponding phosphorylatable amino acid listed in Column D of Table 1), collecting immune serum from the animal, and separating the polyclonal antibodies from the immune serum, in accordance with known procedures. For example, a peptide antigen corresponding to all or part of the novel FYN Proteins kinase phosphorylation site disclosed herein (SEQ ID NO: 17=SLTTGETGYIPRNYVAPVDSIQAEEWyFGK, encompassing phosphorylated tyrosine 150 (see Row 18 of Table 1)) may be employed to produce antibodies that only bind FYN when phosphorylated at Tyr 150. Similarly, a peptide comprising all or part of any one of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85:21-49 (1962)).

It will be appreciated by those of skill in the art that longer or shorter phosphopeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the phosphorylatable amino acid (indicated in Column E by lowercase “y”). Typically, a desirable peptide antigen will comprise four or more amino acids flanking each side of the phosphorylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265:495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal F_{ab} fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The preferred epitope of a phosphorylation-site specific antibody of the invention is a peptide fragment consisting essentially of about 8 to 17 amino acids including the phosphorylatable tyrosine, wherein about 3 to 8 amino acids are positioned on each side of the phosphorylatable tyrosine (for example, the CAMK2G tyrosine 14 phosphorylation site sequence disclosed in Row 5, Column E of Table 1), and antibodies of the invention thus specifically bind a target Brain Ischemia-related signaling polypeptide comprising such epitopic sequence. Particularly preferred epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The invention also provides immortalized cell lines that produce an antibody of the invention. For example, hybridoma clones, constructed as described above, that produce monoclonal antibodies to the Brain Ischemia-related signaling protein phosphorylation sites disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Phosphorylation site-specific antibodies of the invention, whether polyclonal or monoclonal, may be screened for epitope and phospho-specificity according to standard techniques. See, e.g. Czemik et al., Methods in Enzymology, 201: 264-283 (1991). For example, the antibodies may be screened against the phospho and non-phospho peptide library by ELISA to ensure specificity for both the desired antigen (i.e. that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given Brain Ischemia-related signaling protein. The antibodies may also be tested by Western blotting against cell preparations containing the signaling protein, e.g. cell lines over-expressing the target protein, to confirm reactivity with the desired phosphorylated epitope/target.

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the Brain Ischemia-related signaling protein epitope for which the antibody of the invention is specific.

In certain cases, polyclonal antisera may exhibit some undesirable general cross-reactivity to phosphotyrosine or phosphoserine itself, which may be removed by further purification of antisera, e.g. over a phosphotyramine column. Antibodies of the invention specifically bind their target protein (i.e. a protein listed in Column A of Table 1) only when phosphorylated (or only when not phosphorylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to examine Brain Ischemia-related phosphorylation and activation status in diseased tissue. IHC may be carried out according to well-known techniques. See, e.g., ANTIBODIES: A LABORATORY MANUAL, Chapter 10, Harlow & Lane. Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46:72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects a Brain Ischemia-related signal transduction protein enumerated in Table 1), washed and labeled with a fluorescent-labeled secondary antibody. Additional fluorochrome-conjugated marker antibodies (e.g. CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a human Brain Ischemia-related signal transduction protein or polypeptide only when phosphorylated at a disclosed site, but are not limited only to binding the human species, per se. The invention includes antibodies that also bind conserved and highly homologous or identical phosphorylation sites in respective Brain Ischemia-related proteins from other species (e.g. mouse, rat, monkey, yeast), in addition to binding the human phosphorylation site. Highly homologous or identical sites conserved in other species can readily be identified by standard sequence comparisons, such as using BLAST, with the human Brain Ischemia-related signal transduction protein phosphorylation sites disclosed herein.

### C. Heavy-isotope Labeled Peptides (AQUA Peptides).

The novel Brain Ischemia-related signaling protein phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al. and also Gerber et al. Proc. Natl. Acad. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the 99 novel Brain Ischemia-related signaling protein phosphorylation sites disclosed herein (see Table 1/FIG. 2). Peptide standards for a given phosphorylation site (e.g. the tyrosine 13 in CAMK2A—see Row 2 of Table 1) may be produced for both the phosphorylated and non-phosphorylated forms of the site (e.g. see CAMK2A site sequence in Column E, Row 2 of Table 1 (SEQ ID NO: 1) and such standards employed in the AQUA methodology to detect and quantify both forms of such phosphorylation site in a biological sample.

AQUA peptides of the invention may comprise all, or part of, a phosphorylation site peptide sequence disclosed herein (see Column E of Table 1/FIG. 2). In a preferred embodiment, an AQUA peptide of the invention comprises a phosphorylation site sequence disclosed herein in Table 1/FIG. 2. For example, an AQUA peptide of the invention for detection/quantification of DLG4 Adaptor/Scaffold protein when phosphorylated at tyrosine Y758 may comprise the sequence VIEDLSGPyIWVPAR (y=phosphotyrosine), which comprises phosphorylatable tyrosine 758 (see Row 32, Column E; (SEQ ID NO: 31)). Heavy-isotope labeled equivalents of the peptides enumerated in Table 1/FIG. 2 (both in phosphorylated and unphosphorylated form) can be readily synthesized and their unique MS and LC-SRM signature determined, so that the peptides are validated as AQUA peptides and ready for use in quantification experiments.

The phosphorylation site peptide sequences disclosed herein (see Column E of Table 1/FIG. 2) are particularly well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the Brain Ischemia-related phosphorylation sites disclosed in Table 1/FIG. 2 (see Column E) and/or their corresponding parent proteins/polypeptides (see Column A). A phosphopeptide sequence comprising any of the phosphorylation sequences listed in Table 1 may be considered a preferred AQUA peptide of the invention. For example, an AQUA peptide comprising the sequence NVFyELEDVR (SEQ ID NO: 66) (where y may be either phosphotyrosine or tyrosine, and where V=labeled valine (e.g. ¹⁴C)) is provided for the quantification of phosphorylated (or non-phosphorylated) SNIP (Tyr241) in a biological sample (see Row 67 of Table 1, tyrosine 241 being the phosphorylatable residue within the site). However, it will be appreciated that a larger AQUA peptide comprising a disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D of Table 1/FIG. 2) may alternatively be constructed. Such larger or shorter AQUA peptides are within the scope of the present invention, and the selection and production of preferred AQUA peptides may be carried out as described above (see Gygi et al., Gerber et al. supra.).

Certain particularly preferred subsets of AQUA peptides provided by the invention are described above (corresponding to particular protein types/groups in Table 1, for example, Tyrosine Protein Kinases or Protein Phosphatases). Example 4 is provided to further illustrate the construction and use, by standard methods described above, of exemplary AQUA peptides provided by the invention. For example, the above-described AQUA peptides corresponding to both the phosphorylated and non-phosphorylated forms of the disclosed TUBB protein tyrosine 50 phosphorylation site (see Row 71 of Table 1/FIG. 2) may be used to quantify the amount of phosphorylated TUBB (Tyr 50) in a biological sample, e.g. a tumor cell sample (or a sample before or after treatment with a test drug).

AQUA peptides of the invention may also be employed within a kit that comprises one or multiple AQUA peptide(s) provided herein (for the quantification of a Brain Ischemia-related signal transduction protein disclosed in Table 1/FIG. 2), and, optionally, a second detecting reagent conjugated to a detectable group. For example, a kit may include AQUA peptides for both the phosphorylated and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be highly useful in the further study of signal transduction anomalies underlying cancer, including Brain Ischemias, and in identifying diagnostic/bio-markers of these diseases, new potential drug targets, and/or in monitoring the effects of test compounds on Brain Ischemia-related signal transduction proteins and pathways.

### D. Immunoassay Formats

Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target Brain Ischemia-related signal transduction protein is detectable compared to background.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/phosphorylation status of a target Brain Ischemia-related signal transduction protein in patients before, during, and after treatment with a drug targeted at inhibiting phosphorylation of such a protein at the phosphorylation site disclosed herein. For example, bone marrow cells or peripheral blood cells from patients may be analyzed by flow cytometry for target Brain Ischemia-related signal transduction protein phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% para-formaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated Brain Ischemia-related signal transduction protein(s) in the malignant cells and reveal the drug response on the targeted protein.

Alternatively, antibodies of the invention may be employed in immunohistochemical (1HC) staining to detect differences in signal transduction or protein activity using normal and diseased tissues. IHC may be carried out according to well-known techniques. See, e.g., ANTIBODIES: A LABORATORY MANUAL, supra. Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody array formats, such as reversed-phase array applications (see, e.g. Paweletz et al., Oncogene 20(16): 1981-89 (2001)). Accordingly, in another embodiment, the invention provides a method for the multiplex detection of Brain Ischemia-related protein phosphorylation in a biological sample, the method comprising utilizing two or more antibodies or AQUA peptides of the invention to detect the presence of two or more phosphorylated Brain Ischemia-related signaling proteins enumerated in Column A of Table 1/FIG. 2. In one preferred embodiment, two to five antibodies or AQUA peptides of the invention are employed in the method. In another preferred embodiment, six to ten antibodies or AQUA peptides of the invention are employed, while in another preferred embodiment eleven to twenty such reagents are employed.

Antibodies and/or AQUA peptides of the invention may also be employed within a kit that comprises at least one phosphorylation site-specific antibody or AQUA peptide of the invention (which binds to or detects a Brain Ischemia-related signal transduction protein disclosed in Table 1/FIG. 2), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### Example 1

### Isolation of Phosphotyrosine-Containing Peptides from Extracts of Brain Ischemia Cell Lines and Identification of Novel Phosphorylation Sites

In order to discover previously unknown Brain Ischemia-related signal transduction protein phosphorylation sites, IAP isolation techniques were employed to identify phosphotyrosine- and/or phosphoserine-containing peptides in cell extracts from rat brain samples. 10 rats subjected to sham-surgery without ischemia (control), 10 rats subjected to 15 min of ischema and followed by 4 h of repersuion, 10 rats subjected to 20 min of ischema followed by 4 h or reperfusion. Cellular fractions were prepared from these rat brain samples by homogenizing the samples for 50 strokes with a glass-teflon homogeziaer in lysis buffer (TBS, pH7.6, 1 mM sodium orthovanadate, 2.5 mM sodium pyrophosphate). The homogenate was centrifuged at 10,000×g for 20 min at 4 degre to obtain pellet (P2) and supernatant (S2) fractions. P2 pellet was then resuspended with Tris/HCL buffer, pH7.6, 1% Triton X100, 400 mM KCl, 1 mM sodium orthovanadate, and 2.5 mM sodium pyrophosphate. The P2 suspension was then centrifuged at 20,000 g at 4 degree for 10 min to obtain pellet (P2p fraction), and supernatant (P2s fraction). P2p fraction is very rich for phospho-tyrosine signaling, and used for PhosphoScan study.

P2p pellet was lysed in Urea lysis buffer with sonication for 30 sec×2 times, sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used each sample. Columns were washed with 15 volumes of 0.1% TFA, followed by 4 volumes of 5% acetonitrile (MeCN) in 0.1% TFA. Bound peptide was eluted with step-wise increasing concentration of acetonitrile (85, 12%, 15%, 18%, 22%, 25%, 30%, 35%, 40%) in 0.1% TFA. Peptide elute was then lyophilized.

Lyophilized peptide was dissolved in 1.4 ml of IAP buffer (20 mM Tris/HCl or 50 mM MOPS pH 7.2, 10 mM sodium phosphate, 50 mM NaCl) and insoluble matter was removed by centrifugation. The phosphotyrosine monoclonal antibody P-Tyr-100 (Cell Signaling Technology, Inc., catalog number 9411) was coupled at 4 mg/ml beads to protein G or protein A agarose (Roche), respectively. Immobilized antibody (15 μl, 60 μg) was added as 1:1 slurry in IAP buffer to 1.4 ml of cleared peptide solution, and the mixture was incubated overnight at 4° C. with gentle rotation. The immobilized antibody beads were washed three, times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis by LC-MS/MS Mass Spectrometry.

40 μl or more of IAP eluate were purified by 0.2 p, StageTips or ZipTips. Peptides were eluted from the microcolumns with 1 μl of 60% MeCN, 0.1% TFA into 7.6 μl of 0.4% acetic acid/0.005% heptafluorobutyric acid. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LTQ ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (FASTA-formatted protein databases from NCBI: Rat database (Apr. 17, 2005) and Mouse database (May 15, 2005 or Aug. 24, 2004). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned. Furthermore, it should be noted that certain peptides were originally isolated in mouse and later normalized to human sequences as shown by Table 1/FIG. 2.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell. Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the assigned sequences could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria were satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### Example 2

### Production of Phospho-specific Polyclonal Antibodies for the Detection of Brain Ischemia-related Signaling Protein Phosphorylation

Polyclonal antibodies that specifically bind a Brain Ischemia-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, as further described below. Production of exemplary polyclonal antibodies is provided below.

A. CAMK2B (tyrosine 17).

A 13 amino acid phospho-peptide antigen, FTDEYQLY*EDIGK (where y*=phosphotyrosine) that corresponds to the sequence encompassing the tyrosine 17 phosphorylation site in human CAMK2B Protein kinase (see Row 4 of Table 1; SEQ ID NO: 3), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific CAMK2B (tyr17) polyclonal antibodies as described in Immunization/Screening below.

B. CRKL (tyrosine 132).

A 16 amino acid phospho-peptide antigen, TLy*DFPGNDAEDLPFK (where y*=phosphotyrosine) that corresponds to the sequence encompassing the tyrosine 132 phosphorylation site in human CRKL Adaptor/scaffold protein (see Row 24 Table 1 (SEQ ID NO: 23)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific CRKL (tyr 132) polyclonal antibodies as described in Immunization/Screening below.

C. SHB (tyrosine 359).

A 13 amino acid phospho-peptide antigen, AGKGESAGYMEPy*EAQR (where y*=phosphotyrosine) that corresponds to the sequence encompassing the tyrosine 359 phosphorylation site in human PTPRN2 Adaptor/scaffold protein (see Row 41 of Table 1 (SEQ ID NO: 40), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific SHB (tyr 359) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide) are eluted and kept in antibody storage buffer.

The isolated antibody is then tested for phospho-specificity using Western blot assay using an appropriate cell line that expresses (or overexpresses) target phospho-protein (i.e. phosphorylated CAMK2B, CRKL or SHB), for example, rat brain, H1666 and A 431 cells, respectively. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not (substantially) recognize the target protein when not phosphorylated at the appropriate phosphorylation site in the non-stimulated cells (e.g. CRKL is not bound when not phosphorylated attyrosine 132).

In order to confirm the specificity of the isolated antibody, different cell lysates containing various phosphorylated signal transduction proteins other than the target protein are prepared. The Western blot assay is performed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly homologous phosphorylation-site on another protein may be observed. In such case the antibody may be further purified using affinity chromatography, or the specific immunoreactivity cloned by rabbit hybridoma technology.

### Example 3

### Production of Phospho-specific Monoclonal Antibodies for the Detection of Brain Ischemia-related Signaling Protein Phosphorylation

Monoclonal antibodies that specifically bind a Brain Ischemia-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

### A. SHANK2 (Tyrosine 221).

A 14 amino acid phospho-peptide antigen, CFPAASDVNSVy*ER (where y*=phosphotyrosine) that corresponds to the sequence encompassing the tyrosine 221 phosphorylation site in human SHANK2 Adaptor/scaffold protein (see Row 37 of Table 1 (SEQ ID NO: 36)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal SHANK2 (tyr 221) antibodies as described in Immunization/Fusion/Screening below.

### B. GRIN2B (Tyrosine 1039).

An 10 amino acid phospho-peptide antigen, HSQLSDLy*GK (where y*=phosphotyrosine) that corresponds to the sequence encompassing the tyrosine 1039 phosphorylation site in human DDB1-kinase (see Row 52 of Table 1 (SEQ ID NO: 51)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal GRIN2B (tyr1039) antibodies as described in Immunization/Fusion/Screening below.

### C. SYNGAP 1 (Tyrosine 24).

A 16 amino acid phospho-peptide antigen, TQYVHSPy*DRPGWNPR (where y*=phosphotyrosine) that corresponds to the sequence encompassing the tyrosine 24 phosphorylation site in human SYNGAP 1 GTPase (see Row 79 of Table 1 (SEQ ID NO: 78), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal SYNGAP 1 (tyr24) antibodies as described in Immunization/Fusion/Screening below.

### Immunization/Fusion/Screening.

A synthetic phospho-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the SHANK2, GRIN2B or SYNGAP 1 phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. SYNGAP 1 phosphorylated at tyrosine 24).

### Example 4

### Production and Use of AQUA Peptides for the Quantification of Brain Ischemia-related Signaling Protein Phosphorylation

Heavy-isotope labeled peptides (AQUA peptides (internal standards)) for the detection and quantification of a Brain Ischemia-related signal transduction protein only when phosphorylated at the respective phosphorylation site disclosed herein (see Table 1/FIG. 2) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. GNAZ (Tyrosine 154).

An AQUA peptide comprising the sequence, SSEYHLEDNAAY*YLNDLER (y*=phosphotyrosine; sequence incorporating ¹⁴C/¹⁵N-labeled leucine (indicated by bold L), which corresponds to the tyrosine 154 phosphorylation site in human GNAZ G protein (see Row 77 in Table 1 (SEQ ID NO: 76)), is constructed according to standard synthesis techniques using, e.g., a Rainin/Protein Technologies, Inc., Symphony peptide synthesizer (see Merrifield, supra.) as further described below in Synthesis & MS/MS Signature. The GNAZ (tyr 154) AQUA peptide is then spiked into a biological sample to quantify the amount of phosphorylated GNAZ (tyr 154) in the sample, as further described below in Analysis & Quantification.

### B. KCNA2 (Tyrosine 65).

An AQUA peptide comprising the sequence ETEGEEQAQy*LQVTSCPK (y*=phosphotyrosine; sequence incorporating ¹⁴C/¹⁵N-labeled leucine (indicated by bold L), which corresponds to the tyrosine 65 phosphorylation site in human KCNA2 channel protein (see Row 65 in Table 1 (SEQ ID NO: 64)), is constructed according to standard synthesis techniques using, e.g., a Rainin/Protein Technologies, Inc., Symphony peptide synthesizer (see Merrifield, supra.) as further described below in Synthesis & MS/MS Signature. The KCNA2 (tyr65) AQUA peptide is then spiked into a biological sample to quantify the amount of phosphorylated KCNA(tyr65) in the sample, as further described below in Analysis & Quantification.

### C. HPCA (Tyrosine 52)

An AQUA peptide comprising the sequence, Kly*ANFFPYGDASK (y*=phosphotyrosine; sequence incorporating ¹⁴C/¹⁵N-labeled phenylalanine (indicated by bold F), which corresponds to the tyrosine 52 phosphorylation site in human HPCA Calcium binding protein (see Row 49 in Table 1 (SEQ ID NO: 48)), is constructed according to standard synthesis techniques using, e.g., a Rainin/Protein Technologies, Inc., Symphony peptide synthesizer (see Merrifield, supra.) as further described below in Synthesis & MS/MS Signature. The HPCA (tyr52) AQUA peptide is then spiked into a biological sample to quantify the amount of phosphorylated HPCA (tyr52) in the sample, as further described below in Analysis & Quantification.

### D. TOMM34 (Tyrosine 54).

An AQUA peptide comprising the sequence, GSADPEEESVLPGy*SNR (y*=phosphotyrosine; sequence incorporating ¹⁴C/¹⁵N-labeled proline (indicated by bold P), which corresponds to the tyrosine 54 phosphorylation site in human TOMM34 Chaperone protein (see Row 66 in Table 1 (SEQ ID NO:65)), is constructed according to standard synthesis techniques using, e.g., a Rainin/Protein Technologies, Inc., Symphony peptide synthesizer (see Merrifield, supra.) as further described below in Synthesis & MS/MS Signature. The TOMM34 (tyr54) AQUA peptide is then spiked into a biological sample to quantify the amount of phosphorylated TOMM34 (tyr54) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino) methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LTQ ion trap or TSQ Quantum triple quadrupole). On the LTQ, parent ions are isolated at 1.6 m/z width, the ion injection time being limited to 100 ms per microscan, with one microscans per peptide, and with an AGC setting of 1×10⁵; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. An isolated phosphorylation site-specific antibody that specifically binds a human brain ischemia-related signaling protein selected from Column A of Table 1, Rows 14, 2, 25, 29 and 18 only when phosphorylated at the tyrosine listed in corresponding Column D of Table 1, comprised within the phosphorylatable peptide sequence listed in corresponding Column E of Table 1 (SEQ ID NOs: 13, 1, 24, 28 and 17), wherein said antibody does not bind said signaling protein when not phosphorylated at said tyrosine.

35. An isolated phosphorylation site-specific antibody that specifically binds a human brain ischemia-related signaling protein selected from Column A of Table 1, Rows 14, 2, 25, 29 and 18 only when not phosphorylated at the tyrosine listed in corresponding Column D of Table 1, comprised within the phosphorylatable peptide sequence listed in corresponding Column E of Table 1 (SEQ ID NOs: 13, 1, 24, 28 and 17), wherein said antibody does not bind said signaling protein when phosphorylated at said tyrosine.

36. A method selected from the group consisting of:
(a) a method for detecting a human brain ischemia-related signaling protein selected from Column A of Table 1, Rows 14, 2, 25, 29 and 18 wherein said human brain ischemia-related signaling protein is phosphorylated at the tyrosine listed in corresponding Column D of Table 1, comprised within the phosphorylatable peptide sequence listed in corresponding Column E of Table 1 (SEQ ID NOs: 13, 1, 24, 28 and 17), comprising the step of adding an isolated phosphorylation-specific antibody according to claim 34, to a sample comprising said human brain ischemia-related signaling protein under conditions that permit the binding of said antibody to said human brain ischemia-related signaling protein, and detecting bound antibody;
(b) a method for quantifying the amount of a human Brain Ischemia-related signaling protein listed in Column A of Table 1, Rows 14, 2, 25, 29 and 189 that is phosphorylated at the corresponding tyrosine listed in Column D of Table 1, comprised within the phosphorylatable peptide sequence listed in corresponding Column E of Table 1 (SEQ ID NOs: 13, 1, 24, 28 and 17), in a sample using a heavy-isotope labeled peptide (AQUA™ peptide), said labeled peptide comprising a phosphorylated tyrosine at said corresponding tyrosine listed Column D of Table 1, comprised within the phosphorylatable peptide sequence listed in corresponding Column E of Table 1 as an internal standard; and
(c) a method comprising step (a) followed by step (b).

37. The method of claim 36, wherein said isolated phosphorylation-specific antibody is capable of specifically binding PRKCG only when phosphorylated at Y521, comprised within the phosphorylatable peptide sequence listed in Column E, Row 14, of Table 1 (SEQ ID NO: 13), wherein said antibody does not bind said protein when not phosphorylated at said tyrosine.

38. The method of claim 36, wherein said isolated phosphorylation-specific antibody is capable of specifically binding PRKCG only when not phosphorylated at Y521, comprised within the phosphorylatable peptide sequence listed in Column E, Row 14, of Table 1 (SEQ ID NO: 13), wherein said antibody does not bind said protein when phosphorylated at said tyrosine.

39. The method of claim 36, wherein said isolated phosphorylation-specific antibody is capable of specifically binding CAMK2A only when phosphorylated at Y13, comprised within the phosphorylatable peptide sequence listed in Column E, Row 2, of Table 1 (SEQ ID NO: 1), wherein said antibody does not bind said protein when not phosphorylated at said tyrosine.

40. The method of claim 36, wherein said isolated phosphorylation-specific antibody is capable of specifically binding CAMK2A only when not phosphorylated at Y13, comprised within the phosphorylatable peptide sequence listed in Column E, Row 2, of Table 1 (SEQ ID NO: 1), wherein said antibody does not bind said protein when phosphorylated at said tyrosine.

41. The method of claim 36, wherein said isolated phosphorylation-specific antibody is capable of specifically binding DLG1 only when phosphorylated at Y349, comprised within the phosphorylatable peptide sequence listed in Column E, Row 25, of Table 1 (SEQ ID NO: 24), wherein said antibody does not bind said protein when not phosphorylated at said tyrosine.

42. The method of claim 36, wherein said isolated phosphorylation-specific antibody is capable of specifically binding DLG1 only when not phosphorylated at Y349, comprised within the phosphorylatable peptide sequence listed in Column E, Row 25, of Table 1 (SEQ ID NO: 24), wherein said antibody does not bind said protein when phosphorylated at said tyrosine.

43. The method of claim 36, wherein said isolated phosphorylation-specific antibody is capable of specifically binding DLG4 only when phosphorylated at Y150, comprised within the phosphorylatable peptide sequence listed in Column E, Row 29, of Table 1 (SEQ ID NO: 28), wherein said antibody does not bind said protein when not phosphorylated at said tyrosine.

44. The method of claim 36, wherein said isolated phosphorylation-specific antibody is capable of specifically binding DLG4 only when not phosphorylated at Y150, comprised within the phosphorylatable peptide sequence listed in Column E, Row 29, of Table 1 (SEQ ID NO: 28), wherein said antibody does not bind said protein when phosphorylated at said tyrosine.

45. The method of claim 36, wherein said isolated phosphorylation-specific antibody is capable of specifically binding FYN only when phosphorylated at Y150, comprised within the phosphorylatable peptide sequence listed in Column E, Row 18, of Table 1 (SEQ ID NO: 17), wherein said antibody does not bind said protein when not phosphorylated at said tyrosine.

46. The method of claim 36, wherein said isolated phosphorylation-specific antibody is capable of specifically binding FYN only when not phosphorylated at Y150, comprised within the phosphorylatable peptide sequence listed in Column E, Row 18, of Table 1 (SEQ ID NO: 17), wherein said antibody does not bind said protein when phosphorylated at said tyrosine.


SEQUENCE LISTING (100 sequences)

SEQ ID NO: 1 - protein, 13 aa, Homo sapiens
  MOD_RES (5)..(5): Phosphorylated-Tyr
FTEEYQLFEELGK

SEQ ID NO: 2 - protein, 13 aa, Homo sapiens
  MOD_RES (5)..(5): Phosphorylated-Tyr
FTDEYQLYEDIGK

SEQ ID NO: 3 - protein, 13 aa, Homo sapiens
  MOD_RES (8)..(8): Phosphorylated-Tyr
FTDEYQLYEDIGK

SEQ ID NO: 4 - protein, 13 aa, Homo sapiens
  MOD_RES (5)..(5): Phosphorylated-Tyr
FTDDYQLFEELGK

SEQ ID NO: 5 - protein, 21 aa, Homo sapiens
  MOD_RES (6)..(6): Phosphorylated-Tyr
ILAGDYEFDSPYWDDISQAAK

SEQ ID NO: 6 - protein, 26 aa, Homo sapiens
  MOD_RES (9)..(9): Phosphorylated-Tyr
LLNQEEGEYYNVPIPEGDEEGNVELR

SEQ ID NO: 7 - protein, 26 aa, Homo sapiens
  MOD_RES (9)..(9): Phosphorylated-Tyr
LLSQEEGEYFNVPVPPEGSEGNEELR

SEQ ID NO: 8 - protein, 16 aa, Homo sapiens
  MOD_RES (14)..(14): Phosphorylated-Tyr
NLIPMDPNGLSDPYVK

SEQ ID NO: 9 - protein, 25 aa, Homo sapiens
  MOD_RES (9)..(9): Phosphorylated-Tyr
LLNQEEGEYYNVPVADADNCSLLQK

SEQ ID NO: 10 - protein, 25 aa, Homo sapiens
  MOD_RES (10)..(10): Phosphorylated-Tyr
LLNQEEGEYYNVPVADADNCSLLQK

SEQ ID NO: 11 - protein, 13 aa, Homo sapiens
  MOD_RES (6)..(6): Phosphorylated-Tyr
FEACNYPLELYER

SEQ ID NO: 12 - protein, 13 aa, Homo sapiens
  MOD_RES (11)..(11): Phosphorylated-Tyr
FEACNYPLELYER

SEQ ID NO: 13 - protein, 21 aa, Homo sapiens
  MOD_RES (8)..(8): Phosphorylated-Tyr
TFCGTPDYIAPEIIAYQPYGK

SEQ ID NO: 14 - protein, 21 aa, Homo sapiens
  MOD_RES (16)..(16): Phosphorylated-Tyr
TFCGTPDYIAPEIIAYQPYGK

SEQ ID NO: 15 - protein, 21 aa, Homo sapiens
  MOD_RES (19)..(19): Phosphorylated-Tyr
TFCGTPDYIAPEIIAYQPYGK

SEQ ID NO: 16 - protein, 12 aa, Homo sapiens
  MOD_RES (10)..(10): Phosphorylated-Tyr
ANLLANNEYYFR

SEQ ID NO: 17 - protein, 30 aa, Homo sapiens
  MOD_RES (27)..(27): Phosphorylated-Tyr
SLTTGETGYIPSNYVAPVDSIQAEEWYFGK

SEQ ID NO: 18 - protein, 12 aa, Homo sapiens
  MOD_RES (4)..(4): Phosphorylated-Tyr
VCFYSNSFNPGK

SEQ ID NO: 19 - protein, 20 aa, Homo sapiens
  MOD_RES (2)..(2): Phosphorylated-Tyr
TYIDPDTYEDPSLAVHEFAK

SEQ ID NO: 20 - protein, 20 aa, Homo sapiens
  MOD_RES (8)..(8): Phosphorylated-Tyr
TYIDPDTYEDPSLAVHEFAK

SEQ ID NO: 21 - protein, 19 aa, Homo sapiens
  MOD_RES (5)..(5): Phosphorylated-Tyr
TVPDYTTFTTVGDWLDAIK

SEQ ID NO: 22 - protein, 25 aa, Homo sapiens
  MOD_RES (11)..(11): Phosphorylated-Tyr
DHLVPSTHYIYEEPEVQSGDVSYPR

SEQ ID NO: 23 - protein, 16 aa, Homo sapiens
  MOD_RES (3)..(3): Phosphorylated-Tyr
TLYDFPGNDAEDLPFK

SEQ ID NO: 24 - protein, 25 aa, Homo sapiens
  MOD_RES (22)..(22): Phosphorylated-Tyr
GLGFSIAGGVGNQHIPGDNSIYVTK

SEQ ID NO: 25 - protein, 21 aa, Homo sapiens
  MOD_RES (8)..(8): Phosphorylated-Tyr
HMLVEDDYTRPPEPVYSTVNK

SEQ ID NO: 26 - protein, 20 aa, Homo sapiens
  MOD_RES (12)..(12): Phosphorylated-Tyr
FIEAGQYNDNLYGTSVQSVR

SEQ ID NO: 27 - protein, 9 aa, Homo sapiens
  MOD_RES (3)..(3): Phosphorylated-Tyr
NTYDVVYLK

SEQ ID NO: 28 - protein, 9 aa, Homo sapiens
  MOD_RES (7)..(7): Phosphorylated-Tyr
NTYDVVYLK

SEQ ID NO: 29 - protein, 20 aa, Homo sapiens
  MOD_RES (7)..(7): Phosphorylated-Tyr
FIEAGQYNSHLYGTSVQSVR

SEQ ID NO: 30 - protein, 20 aa, Homo sapiens
  MOD_RES (12)..(12): Phosphorylated-Tyr
FIEAGQYNSHLYGTSVQSVR

SEQ ID NO: 31 - protein, 15 aa, Homo sapiens
  MOD_RES (9)..(9): Phosphorylated-Tyr
VIEDLSGPYIWVPAR

SEQ ID NO: 32 - protein, 14 aa, Homo sapiens
  MOD_RES (4)..(4): Phosphorylated-Tyr
GGLYNSMDSLDSNK

SEQ ID NO: 33 - protein, 10 aa, Homo sapiens
  MOD_RES (4)..(4): Phosphorylated-Tyr
NENYFDDIPR

SEQ ID NO: 34 - protein, 15 aa, Homo sapiens
  MOD_RES (7)..(7): Phosphorylated-Tyr
YLEHGEYEGNLYGTR

SEQ ID NO: 35 - protein, 15 aa, Homo sapiens
  MOD_RES (12)..(12): Phosphorylated-Tyr
YLEHGEYEGNLYGTR

SEQ ID NO: 36 - protein, 14 aa, Homo sapiens
  MOD_RES (12)..(12): Phosphorylated-Tyr
CFPAASDVNSVYER

SEQ ID NO: 37 - protein, 13 aa, Homo sapiens
  MOD_RES (8)..(8): Phosphorylated-Tyr
GQMPENPYSEVGK

SEQ ID NO: 38 - protein, 23 aa, Homo sapiens
  MOD_RES (9)..(9): Phosphorylated-Tyr
LGAGAAGLYDSGTPLGPLPYPER

SEQ ID NO: 39 - protein, 26 aa, Homo sapiens
  MOD_RES (8)..(8): Phosphorylated-Tyr
PSGPDSPYANLGAFSASLFAPSKPQR

SEQ ID NO: 40 - protein, 17 aa, Homo sapiens
  MOD_RES (13)..(13): Phosphorylated-Tyr
AGKGESAGYMEPYEAQR

SEQ ID NO: 41 - protein, 15 aa, Homo sapiens
  MOD_RES (6)..(6): Phosphorylated-Tyr
IAAAQYSVTGSAVAR

SEQ ID NO: 42 - protein, 16 aa, Homo sapiens
  MOD_RES (13)..(13): Phosphorylated-Tyr
ALQSPEHHIDPIYEDR

SEQ ID NO: 43 - protein, 16 aa, Homo sapiens
  MOD_RES (11)..(11): Phosphorylated-Tyr
ASYAAGPASNYADPYR

SEQ ID NO: 44 - protein, 16 aa, Homo sapiens
  MOD_RES (15)..(15): Phosphorylated-Tyr
ASYAAGPASNYADPYR

SEQ ID NO: 45 - protein, 13 aa, Homo sapiens
  MOD_RES (2)..(2): Phosphorylated-Tyr
DYETYQPFQNSTR

SEQ ID NO: 46 - protein, 13 aa, Homo sapiens
  MOD_RES (5)..(5): Phosphorylated-Tyr
DYETYQPFQNSTR

SEQ ID NO: 47 - protein, 18 aa, Homo sapiens
  MOD_RES (13)..(13): Phosphorylated-Tyr
TVHDMDQFGQQQYDIYER

SEQ ID NO: 48 - protein, 14 aa, Homo sapiens
  MOD_RES (3)..(3): Phosphorylated-Tyr
KIYANFFPYGDASK

SEQ ID NO: 49 - protein, 17 aa, Homo sapiens
  MOD_RES (13)..(13): Phosphorylated-Tyr
GFNTLPSTEISMYTLSR

SEQ ID NO: 50 - protein, 24 aa, Homo sapiens
  MOD_RES (20)..(20): Phosphorylated-Tyr
DASPGGPGGPGFASTDISMYTLSR

SEQ ID NO: 51 - protein, 10 aa, Homo sapiens
  MOD_RES (8)..(8): Phosphorylated-Tyr
HSQLSDLYGK

SEQ ID NO: 52 - protein, 22 aa, Homo sapiens
  MOD_RES (13)..(13): Phosphorylated-Tyr
SDVSDISTHTVTYGNIEGNAAK

SEQ ID NO: 53 - protein, 10 aa, Homo sapiens
  MOD_RES (9)..(9): Phosphorylated-Tyr
EFDEIELAYR

SEQ ID NO: 54 - protein, 18 aa, Homo sapiens
  MOD_RES (15)..(15): Phosphorylated-Tyr
ENSPHWEHVDLTDIYKER

SEQ ID NO: 55 - protein, 21 aa, Homo sapiens
  MOD_RES (15)..(15): Phosphorylated-Tyr
SYNNPPCEENLFSDYISEVER

SEQ ID NO: 56 - protein, 17 aa, Homo sapiens
  MOD_RES (4)..(4): Phosphorylated-Tyr
NNTYAPTATSYTPNLAR

SEQ ID NO: 57 - protein, 30 aa, Homo sapiens
  MOD_RES (26)..(26): Phosphorylated-Tyr
AESKRMKLTKNTQNFKPAPATNTQNYATYR

SEQ ID NO: 58 - protein, 20 aa, Homo sapiens
  MOD_RES (19)..(19): Phosphorylated-Tyr
NTQNFKPAPATNTQNYATYR

SEQ ID NO: 59 - protein, 12 aa, Homo sapiens
  MOD_RES (6)..(6): Phosphorylated-Tyr
EGYNVYGTESVK

SEQ ID NO: 60 - protein, 20 aa, Homo sapiens
  MOD_RES (16)..(16): Phosphorylated-Tyr
GSLIVDMVSDKGNLIYSDNR

SEQ ID NO: 61 - protein, 18 aa, Homo sapiens
  MOD_RES (4)..(4): Phosphorylated-Tyr
EEVYQQDWSQNNALQFQK

SEQ ID NO: 62 - protein, 17 aa, Homo sapiens
  MOD_RES (7)..(7): Phosphorylated-Tyr
LLEGNLYGSLFSVPSSK

SEQ ID NO: 63 - protein, 22 aa, Homo sapiens
  MOD_RES (5)..(5): Phosphorylated-Tyr
MGNLYDIDEDQMLQETGNPATR

SEQ ID NO: 64 - protein, 18 aa, Homo sapiens
  MOD_RES (10)..(10): Phosphorylated-Tyr
ETEGEEQAQYLQVTSCPK

SEQ ID NO: 65 - protein, 15 aa, Homo sapiens
  MOD_RES (12)..(12): Phosphorylated-Tyr
GSADPEEESVLYSNR

SEQ ID NO: 66 - protein, 10 aa, Homo sapiens
  MOD_RES (4)..(4): Phosphorylated-Tyr
NVFYELEDVR

SEQ ID NO: 67 - protein, 18 aa, Homo sapiens
  MOD_RES (4)..(4): Phosphorylated-Tyr
EPLYAAFPGSHLTNGDLR

SEQ ID NO: 68 - protein, 16 aa, Homo sapiens
  MOD_RES (5)..(5): Phosphorylated-Tyr
GEGLYADPYGLLHEGR

SEQ ID NO: 69 - protein, 16 aa, Homo sapiens
  MOD_RES (9)..(9): Phosphorylated-Tyr
GEGLYADPYGLLHEGR

SEQ ID NO: 70 - protein, 12 aa, Homo sapiens
  MOD_RES (4)..(4): Phosphorylated-Tyr
ISVYYNEATGGK

SEQ ID NO: 71 - protein, 21 aa, Homo sapiens
  MOD_RES (9)..(9): Phosphorylated-Tyr
DSSALQDLYIPPPPAEPYIPR

SEQ ID NO: 72 - protein, 21 aa, Homo sapiens
  MOD_RES (10)..(10): Phosphorylated-Tyr
LRPISMPVEYNWVGDYEDPNK

SEQ ID NO: 73 - protein, 21 aa, Homo sapiens
  MOD_RES (16)..(16): Phosphorylated-Tyr
LRPISMPVEYNWVGDYEDPNK

SEQ ID NO: 74 - protein, 19 aa, Homo sapiens
  MOD_RES (7)..(7): Phosphorylated-Tyr
QYKPVVYSNTIQSLAAIVR

SEQ ID NO: 75 - protein, 19 aa, Homo sapiens
  MOD_RES (4)..(4): Phosphorylated-Tyr
SSEYHLEDNAAYYLNDLER

SEQ ID NO: 76 - protein, 19 aa, Homo sapiens
  MOD_RES (12)..(12): Phosphorylated-Tyr
SSEYHLEDNAAYYLNDLER

SEQ ID NO: 77 - protein, 16 aa, Homo sapiens
  MOD_RES (3)..(3): Phosphorylated-Tyr
TQYVHSPYDRPGWNPR

SEQ ID NO: 78 - protein, 16 aa, Homo sapiens
  MOD_RES (8)..(8): Phosphorylated-Tyr
TQYVHSPYDRPGWNPR

SEQ ID NO: 79 - protein, 12 aa, Homo sapiens
  MOD_RES (4)..(4): Phosphorylated-Tyr
DLFYVSRPPLAR

SEQ ID NO: 80 - protein, 18 aa, Homo sapiens
  MOD_RES (5)..(5): Phosphorylated-Tyr
SSPAYCTSSSDITEPEQK

SEQ ID NO: 81 - protein, 17 aa, Homo sapiens
  MOD_RES (15)..(15): Phosphorylated-Tyr
GGEPPGDTFAPFHGYSK

SEQ ID NO: 82 - protein, 35 aa, Homo sapiens
  MOD_RES (22)..(22): Phosphorylated-Tyr
SEDLSTGVPKPPAASILHSHSYSDEFGPSGTDFTR

SEQ ID NO: 83 - protein, 20 aa, Homo sapiens
  MOD_RES (14)..(14): Phosphorylated-Tyr
PSSGNLLQSPEPSYGPARPR

SEQ ID NO: 84 - protein, 22 aa, Homo sapiens
  MOD_RES (13)..(13): Phosphorylated-Tyr
TAAFLLPILSQIYADGPGEALR

SEQ ID NO: 85 - protein, 25 aa, Homo sapiens
  MOD_RES (24)..(24): Phosphorylated-Tyr
YIHRVGRTARAGRSGKAITFVTQYD

SEQ ID NO: 86 - protein, 12 aa, Homo sapiens
  MOD_RES (4)..(4): Phosphorylated-Tyr
INVYYNEATGGK

SEQ ID NO: 87 - protein, 12 aa, Homo sapiens
  MOD_RES (4)..(4): Phosphorylated-Tyr
INVYYNEAAGNK

SEQ ID NO: 88 - protein, 15 aa, Homo sapiens
  MOD_RES (10)..(10): Phosphorylated-Tyr
PSEPGSEGDYMVLPR

SEQ ID NO: 89 - protein, 19 aa, Homo sapiens
  MOD_RES (8)..(8): Phosphorylated-Tyr
TPSEYPHYTTINVLDTEAK

SEQ ID NO: 90 - protein, 11 aa, Homo sapiens
  MOD_RES (6)..(6): Phosphorylated-Tyr
MMESDYIVMPR

SEQ ID NO: 91 - protein, 23 aa, Homo sapiens
  MOD_RES (18)..(18): Phosphorylated-Tyr
LPPIPYPNAPCFNNVAEYESFLK

SEQ ID NO: 92 - protein, 17 aa, Homo sapiens
  MOD_RES (8)..(8): Phosphorylated-Tyr
AFVAIGDYNGHVGLGVK

SEQ ID NO: 93 - protein, 29 aa, Homo sapiens
  MOD_RES (8)..(8): Phosphorylated-Tyr
ELPTEPPYTAYVGNLPFNTVQGDIDAIFK

SEQ ID NO: 94 - protein, 18 aa, Homo sapiens
  MOD_RES (15)..(15): Phosphorylated-Tyr
QKEPKELKLSVPMPYMLK

SEQ ID NO: 95 - protein, 21 aa, Homo sapiens
  MOD_RES (9)..(9): Phosphorylated-Tyr
ESNSNQCVYAAHNSVVIDECK

SEQ ID NO: 96 - protein, 20 aa, Homo sapiens
  MOD_RES (17)..(17): Phosphorylated-Tyr
TSCTGGPQLFGYPDPTYLTR

SEQ ID NO: 97 - protein, 18 aa, Homo sapiens
  MOD_RES (5)..(5): Phosphorylated-Tyr
SSGDYSATFLPGLIPAEK

SEQ ID NO: 98 - protein, 18 aa, Homo sapiens
  MOD_RES (3)..(3): Phosphorylated-Tyr
RIYTLDPYPMDLYHPDQR

SEQ ID NO: 99 - protein, 13 aa, Homo sapiens
  MOD_RES (5)..(5): Phosphorylated-Tyr
LHQVYFDAPSCVK

SEQ ID NO: 100 - protein, 16 aa, Homo sapiens
  MOD_RES (3)..(3): Phosphorylated-Tyr
TQYVHSPYDRPGWNPR